(19) United States
(12) Patent Application Publication
(10) Pub. No.: US 2025/0161310 A1
(43) Pub. Date: May 22, 2025

(54) PHARMACEUTICAL COMBINATIONS FOR USE IN THE TREATMENT OF NEOPLASTIC DISEASES

(71) Applicant: SILLAJEN, INC., Seoul (KR)

(72) Inventors: Heidi LANE, Allschwil (CH); Paul MCSHEEHY, Allschwil (CH); Nicole FORSTER-GROSS, Allschwil (CH)

(21) Appl. No.: 18/841,770

(22) PCT Filed: Mar. 3, 2023
(86) PCT No.: PCT/KR2023/002954

§ 371 Date: Aug. 27, 2024

(30) Foreign Application Priority Data
Mar. 4, 2022 (EP) 22160244.4
Aug. 22, 2022 (EP) 22191430.2

(51) Int. Cl. A61K 31/519; A61K 31/555; A61P 35/00

(52) U.S. Cl. CPC A61K 31/519; A61K 31/555; A61P 35/00

(57) ABSTRACT

The present invention provides pharmaceutical combinations comprising (a) a TTK/PLK1 inhibitor (i.e. a compound of formula (I) or a pharmaceutically acceptable salt thereof as defend in the claims) and (b) carboplatin, as well as methods of using the combinations of the invention for the treatment of neoplastic diseases such as cancer.

### TECHNICAL FIELD

The present invention relates to pharmaceutical combinations comprising two active pharmaceutical compounds as described herein and methods of using the combinations of the invention in the treatment of neoplastic diseases, in particular cancer.

### BACKGROUND ART

WO 2015/155042 describes a recently discovered class of inhibitors of the threonine tyrosine kinase (TTK) for use in the treatment of cancer.

Carboplatin is a well-known small molecule approved for the treatment of a number of cancer indications. It is commercialized under the brand name Paraplatin®.

There is an ongoing need for new effective treatment options for cancer patients.

### DISCLOSURE OF INVENTION

### Solution to Problem

### Summary of the Invention

In a first aspect the present invention provides a pharmaceutical combination comprising
(a) a compound of formula (I)

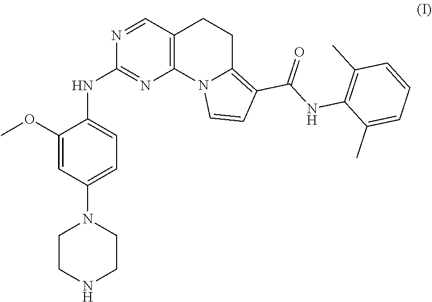 [SMILES: COc1cc(N2CCNCC2)ccc1Nc1ncc2c(n1)-n1ccc(C(=O)Nc3c(C)cccc3C)c1CC2]

or a pharmaceutically acceptable salt thereof;
and (b) carboplatin.

The compound of formula (I) is disclosed in WO 2015/155042 as Example 17.

It has surprisingly been found that in addition to TTK inhibitory activity the compound of formula (I) also exhibits polo-like kinase 1 (PLK1) inhibition (see the Examples below). Both kinases collaborate in activating the mitotic spindle assembly checkpoint (SAC) at the kinetochore to regulate chromosome alignment and segregation before the cell can exit mitosis. Von Schubert et al., Cell Reports 2015, 12; 66-78 discloses that PLK1 and TTK (also known as MPS1) cooperatively regulate the spindle assembly checkpoint (SAC) in human cells. This potential for an enhanced effect from inhibition of both TTK and PLK1 is also alluded to in Dou et al., Plos ONE 2011, 6:4; e18793, which shows that some substrates of both kinases share similar consensus motifs.

In contrast to TTK-specific inhibitors, the compound of formula (I) has a prolonged effect on TTK combined with a transient effect on PLK1 (see Examples below) leading to a more rapid disruption of the SAC that potentiates aberrant mitotic progression. Accordingly, the dual TTK/PLK1 inhibitory activity gives the compound of formula (I) a unique profile and differentiates it from other molecules which show TTK inhibitory activity without any appreciable levels of PLK1 inhibitory activity.

In a further aspect the invention provides a method for treating a neoplastic disease in a subject in need thereof comprising administering to the subject a therapeutically effective amount of the pharmaceutical combination of the invention.

In a further aspect the invention provides a method for treating a neoplastic disease in a subject in need thereof comprising administering to the subject a therapeutically effective amount of the compound of formula (I) or a pharmaceutically acceptable salt thereof, wherein said subject is undergoing or will undergo treatment with carboplatin.

In a further aspect the invention provides a method for treating a neoplastic disease in a subject in need thereof comprising administering to the subject a therapeutically effective amount of carboplatin, wherein said subject is undergoing or will undergo treatment with the compound of formula (I) or a pharmaceutically acceptable salt thereof.

In a further aspect the invention provides the pharmaceutical combination of the invention for use in the treatment of a neoplastic disease in a subject.

In a further aspect the invention provides a compound of formula (I) or a pharmaceutically acceptable salt thereof for use in combination with carboplatin for the treatment of a neoplastic disease in a subject.

In a further aspect the invention provides carboplatin for use in combination with a compound of formula (I) or a pharmaceutically acceptable salt thereof for the treatment of a neoplastic disease in a subject.

In a further aspect the invention provides use of the pharmaceutical combination of the invention in the preparation of single-agent medicaments or as a combined medicament for the treatment of a neoplastic disease in a subject.

In a further aspect the invention provides use of a compound of formula (I) or a pharmaceutically acceptable salt thereof in the preparation of a single-agent medicament for use in combination with carboplatin, or in the preparation of a combined medicament with carboplatin, for the treatment of a neoplastic disease in a subject.

In a further aspect the invention provides use of carboplatin in the preparation of a single-agent medicament for use in combination with a compound of formula (I) or a pharmaceutically acceptable salt thereof or in the preparation of a combined medicament with the compound of formula (I) or a pharmaceutically acceptable salt thereof, for the treatment of a neoplastic disease in a subject.

Neoplastic diseases for treatment by combinations of the invention are described below, and are in particular contemplated for treatment of cancer, and in particular for human subjects.

Additional aspects and embodiments of the invention are described in more detail below.

### BRIEF DESCRIPTION OF FIGURES

FIG. 1: FIG. 1 shows additivity of compound of formula (I) in combination with carboplatin in the TNBC PDX model BR1282. Carboplatin (60 mg/kg) was administered weekly (qw) i.v. The compound of formula (I) (7 mg/kg) was administered twice-weekly (2qw) i.v. (4 or 24 hours after carboplatin, when administered on the same day). Mean tumor volume (A) and mean body weight changes in % (B) are shown. One non-drug-related death in the vehicle-treated group occurred on day 15. One mouse in the combination group with the 4 hour dosing gap on day 20 and in the 24 hour dosing gap on day 10 had to be euthanized due to tumor volume above 1500 mm3. Dosing was delayed by 1 day in any mice showing transient body weight loss of ≥10% at any time point. The vehicle control group received the vehicle of carboplatin and the vehicle of the compound of formula (I) using a 4 h gap between both administrations (indicated as both vehicles). N=8 mice per group.

FIG. 2: FIG. 2 shows the individual tumors of the study presented in FIG. 1 until sacrifice. The vehicle control group received the vehicle of carboplatin and the vehicle of the compound of formula (I) using a 4 h gap between both administrations (indicated as both vehicles).

FIG. 3: FIG. 3 shows the mean rate of growth (k) suggesting that the combinations of carboplatin and the compound of formula (I) have additive efficacy as the single agents in the BR1282 TNBC PDX model. Shown are the exponential cal-culations of k means of treatment with both vehicles (vehicle), 7 mg/kg of the compound of formula (I) i.v. 2qw, 60 mg/kg carboplatin i.v. qw or a combination of both until the day of sacrificing the mice (latest day 31). In the combination groups, carboplatin was administered first, followed by the compound of formula (I) 4 or 24 hours later when administered on the same day. The line represents the mean+/−SD. Statistical analysis is based on one-way-analysis of variance (1WA) with Holm-Sidak post-hoc for group comparisons. All groups treated with the different compounds were statistically significantly different compared to the vehicle, and both combination groups are statistically different compared to the carboplatin single agent group (p<0.05). However, the combination groups are not statistically different compared to the compound of formula (I) single agent group (p=0.5736). Detailed statistical analysis in Table 2. N=8 mice per group.

FIG. 4: FIG. 4 shows antagonism of compound of formula (I) in combination with carboplatin in the TNBC PDX model BR1282, when compound of formula (I) was administered first and carboplatin second. The compound of formula (I) (8 mg/kg) and carboplatin (60 mg/kg) were administered qw i.v. (carboplatin 4 hours after compound of formula (I), when administered on the same day). Mean tumor volume (A) and mean body weight changes in % (B) are shown. The vehicle control group received the vehicle of the compound of formula (I) and the vehicle of carboplatin using a 4 h gap between both administrations (indicated as both vehicles). N=6 mice for vehicle and both single agent groups, N=7 mice in the combination group.

FIG. 5: FIG. 5 shows the individual tumors of the study presented in FIG. 4. The vehicle control group received the vehicle of the compound of formula (I) and the vehicle of carboplatin using a 4 h gap between both administrations (indicated as both vehicles).

FIG. 6: FIG. 6 shows the statistical analysis at day 14 suggesting that the combination of carboplatin and the compound of formula (I) have similar efficacy as the single agents in the BR1282 TNBC PDX model. Shown are the A tumor volumes (tumor volume on day 14−tumor volume on day 0) on day 14 of treatment with both vehicles (vehicle), 8 mg/kg of the compound of formula (I) i.v. qw, 60 mg/kg carboplatin i.v. qw or a combination of both. In the combination groups, the compound of formula (I) was administered first, followed by carboplatin 4 hours later when administered on the same day. The line represents the mean+/−SD. Statistical analysis is based on one-way-analysis of variance (1WA) with Holm-Sidak post-hoc for group comparisons. All groups treated with the different compounds were not statistically significantly different compared to the vehicle, and the combination group was not statistically different as compared to the compound of formula (I) single agent group (p>0.05). N=6 mice for vehicle and both single agent groups, N=7 mice in the combination group.

FIG. 7: FIG. 7 shows efficacy (A) and tolerability (B) of the combination of carboplatin plus the compound of formula (I) in Balb/c nude mice bearing SKOV-3 s.c. xenografts. Balb-c nude mice bearing s.c. SKOV-3 tumors were treated with the single-agents or combinations at the doses and schedules shown until day 24. The time-dependent changes in tumor volume (efficacy) and body-weight (tolerability) show the mean±SEM (n=8), until individual mice needed to be culled due to their tumor size exceeding 1500 mm³. “CRB” refers to carboplatin.

FIG. 8: FIG. 8 shows efficacy (A) and tolerability (B) of the combination of carboplatin plus the compound of formula (I) in Balb/c nude mice bearing SKOV-3 s.c. xenografts until first culling due to excessive tumor size. Balb-c nude mice bearing s.c. SKOV-3 tumors were treated with the single-agents or combinations at the doses and schedules shown.

FIG. 9: FIG. 9 shows endpoint statistical analysis at day 23 for tumor volume (A) and body weight (B) using a one-way ANOVA with Holm-Sidak post-hoc for multiple comparisons, where *p<0.05, **p<0.01, ***p<0.001 compared to the vehicle group.

FIG. 10: FIG. 10 shows exponential fits to individual SKOV-3 tumors in each treatment group. Since mice needed to be culled regularly until the study end on day 51, an alternative presentation is shown with the exponential fits (Prism 9.3) for each tumor in each group from day 0 until the last day of measurement. “Combination” refers to combination treatment using the full doses of both compounds with the compound of formula (I) on a 2qw or qw schedule with an interval of 1, 4 or 24 hr as indicated.

### DETAILED DESCRIPTION OF THE INVENTION

### Definitions

Certain terms used herein are described below. Unless defined otherwise, all technical and scientific terms used herein have the same meaning as is commonly understood by one of skill in the art to which this invention belongs.

The term “combination”, “therapeutic combination”, or “pharmaceutical combination” are interchangeable terms and refer to either a fixed combination or a non-fixed combination.

The term “combination therapy” refers to the administration of two or more therapeutic agents to treat a therapeutic condition or disorder described in the present disclosure. Such administration encompasses co-administration of these therapeutic agents in a substantially simultaneous manner as well as use of each type of therapeutic agent in a sequential and/or separate manner (e.g. according to different administration routes), either at approximately the same time or at different times, e.g. according to different dosage regimens, examples of which are described herein. When the therapeutic agents are administered sequentially and/or separately the dosing schedules will be such that there is a therapeutic interaction between the therapeutic agents within the patient's body and/or that a therapeutic effect resulting from the first therapeutic agent is present when the second therapeutic agent is administered. For example, when the agents are administered according to cyclic treatment schedules, the cyclic treatment schedules may overlap, or when one therapeutic agent is administered according to a continuous dosing schedule and the second according to a cyclic schedule, then at least one dose from the agent administered according to the continuous schedule will occur during the treatment cycle of the other therapeutic agent. Usually there will be at least one interval of no more than seven days between a dose of one of the therapeutic agents and a dose of the other therapeutic agent when both therapeutic agents are administered according to cyclic treatment schedules.

The term “pharmaceutical composition” is defined herein to refer to a solid or liquid formulation containing at least one therapeutic agent to be administered to a patient, optionally with one or more pharmaceutically acceptable excipients, in order to treat a particular disease or condition affecting the patient.

The term “pharmaceutically acceptable” as used herein refers to items such as compounds, materials, compositions and/or dosage forms, which are, within the scope of sound medical judgment, suitable for contact with the tissues of a human, without excessive toxicity or other complications commensurate with a reasonable benefit/risk ratio.

The terms “fixed combination”, “fixed dose”, “single formulation” and “combined medicament” as used herein refers to a single dosage form formulated to deliver an amount, which is jointly therapeutically effective for the treatment of neoplastic diseases, of both therapeutic agents to a patient. The single dosage form is designed to deliver an amount of each of the therapeutic agents, along with any pharmaceutically acceptable carriers or excipients.

The term “non-fixed combination,”, “kit”, “separate formulations” and “single-agent medicaments” means that the active ingredients are formulated as separate entities to allow administration to a patient either simultaneously, sequentially or separately, wherein such administration provides therapeutically effective levels of the two compounds in the body of the patient.

The term “patient” refers to a human presenting themselves for therapeutic treatment.

The term “subject” refers to a mammal and preferably refers to a patient.

The term “treatment,” as used herein in the context of treating a disease in a subject pertains generally to treatment and therapy in which some desired therapeutic effect is achieved, for example one or more of the following: the inhibition of the progress of the disease, a reduction in the rate of progress, a halt in the rate of progress, a prevention of the progression of the disease, alleviation of symptoms of the disease, amelioration of disease, and cure of the disease. For example, treatment can be the di-minishment of one or several symptoms of a disorder or complete eradication of a disorder. Within the meaning of the present disclosure, the term “treat” also denotes to arrest, delay the onset (i.e. the period prior to clinical manifestation of a disease) and/or reduce the risk of developing or worsening of a disease.

The term “prevent”, “preventing” or “prevention” as used herein comprises the prevention of at least one symptom associated with or caused by disease being prevented.

The term “pharmaceutically effective amount,” “therapeutically effective amount,” or “clinically effective amount” is an amount sufficient to provide an observable or clinically significant improvement over the baseline clinically observable signs and symptoms of the disease treated, e.g. commensurate with a reasonable benefit/risk ratio, when administered in accordance with a desired treatment regimen. The skilled person will understand that the therapeutically effective amount of an agent for use in combination therapy may be lower than the amount required to provide a therapeutic effect when using the agent as a monotherapy.

The term “about” means a variation of no more than 10% of the relevant figure. In some embodiments the term “about” means a variation of no more than 5% of the relevant figure.

For the avoidance of doubt, where a range is provided (e.g. 5 mg to 480 mg) the range includes the stated upper limit (480 mg) and lower limit (5 mg) of the range.

### Compounds of Formula (I)

In some embodiments the compound of formula (I) is/is used as the free base. In other embodiments the compound of formula (I) is used as a pharmaceutically acceptable salt.

Pharmaceutically acceptable salts of the compound of formula (I) may be acid addition salts. Salts are formed e.g. with organic or inorganic acids from compounds of formula (I). Pharmaceutically acceptable salts are within the common general knowledge of the person skilled in the art. Pharmaceutically acceptable salts may include more than one molecule or ion of the corresponding acid.

The compounds of formula (I) and pharmaceutically acceptable salts thereof may be solvated, especially hydrated. Solvation and/or hydration may take place during the preparation process.

Compounds of formula (I) and pharmaceutically acceptable salts thereof may be syn-thesized as described in WO 2015/155042, in particular on pages 17 to 19 which are hereby incorporated by reference, and as described in Example 17 on page 49 of WO 2015/155042, which is also hereby incorporated by reference, including the reference in Example 17 to Example 9, Intermediate H and Example 1.

### Diseases

The pharmaceutical combinations of the invention may be used to treat neoplastic diseases by administration of the combinations of the invention, e.g. to inhibit the protein kinase TTK (compound of formula (I) or a pharmaceutically acceptable salt thereof) and inhibit DNA repair and/or synthesis (carboplatin). In addition in view of the observation that compounds of formula (I) additionally have PLK1 inhibitory activity the neoplastic disease may be one which is treatable by inhibition of PLK1 in addition to a treatment with carboplatin and a TTK inhibitor (e.g. the compound of formula (I)).

In addition, the pharmaceutical combinations of the invention may be used to treat a cancer at any clinical stage or pathological grade (e.g. tumor stage I, tumor stage II, tumor stage III, tumor stage IV) or treatment settings (e.g. preventative, adjuvant, neoadjuvant, therapeutic including palliative treatment). The pharmaceutical combinations of the invention may be for use in slowing, delaying or stopping cancer progression or cancer growth or increasing the overall survival time or the cancer-progression-free survival time or the time to progression of a cancer or improving or maintaining the subject's (e.g. patient's) quality of life or functional status. The phar-maceutical combinations of the invention may also be used in post-therapy recovery from cancer. The pharmaceutical combinations of the invention may be used in the treatment of metastatic cancer. The cancer to be treated may be an advanced cancer. An advanced cancer is one that is unlikely to be cured or controlled with treatment. The cancer may have spread from where it first started to nearby tissue, lymph nodes, or distant parts of the body. In this case treatment may be given to help shrink the tumor, slow the growth of cancer cells, or relieve symptoms.

For example, the pharmaceutical combinations of the invention may be used for (i) reducing the number of cancer cells; (ii) reducing tumor volume; (iii) increasing tumor regression rate; (iv) reducing or slowing cancer cell infiltration into peripheral organs; (v) reducing or slowing tumor metastasis; (vi) reducing or inhibiting tumor growth; (vii) preventing or delaying occurrence and/or recurrence of the cancer and/or extends disease- or tumor-free survival time; (viii) increasing overall survival time; (ix) reducing the frequency of treatment; and/or (x) relieving one or more of symptoms associated with the cancer.

As mentioned above, the pharmaceutical combinations of the invention may be used for the treatment of neoplastic diseases. Examples of neoplastic diseases include, but are not limited to, epithelial neoplasms, squamous cell neoplasms, basal cell neoplasms, transitional cell papillomas and carcinomas, adenomas and adeno-carcinomas, adnexal and skin appendage neoplasms, mucoepidermoid neoplasms, cystic neoplasms, mucinous and serous neoplasms, ducal-, lobular and medullary neoplasms, acinar cell neoplasms, complex epithelial neoplasms, specialized gonadal neoplasms, paragangliomas and glomus tumors, naevi and melanomas, soft tissue tumors and sarcomas, fibromatous neoplasms, myxomatous neoplasms, lipomatous neoplasms, myomatous neoplasms, complex mixed and stromal neoplasms, fibroep-ithelial neoplasms, synovial like neoplasms, mesothelial neoplasms, germ cell neoplasms, trophoblastic neoplasms, mesonephromas, blood vessel tumors, lymphatic vessel tumors, osseous and chondromatous neoplasms, giant cell tumors, mis-cellaneous bone tumors, odontogenic tumors, gliomas, neuroepitheliomatous neoplasms, meningiomas, nerve sheath tumors, granular cell tumors and alveolar soft part sarcomas, Hodgkin's and non-Hodgkin's lymphomas, other lymphoreticular neoplasms, plasma cell tumors, mast cell tumors, immunoproliferative diseases, leukemias, miscellaneous myeloproliferative disorders, lymphoproliferative disorders and myelodysplastic syndromes.

In some embodiments the neoplastic disease is cancer. Examples of cancers in terms of the organs and parts of the body affected include, but are not limited to, the brain, breast (including triple negative breast cancer), cervix, ovaries, colon, rectum (including colon and rectum i.e. colorectal cancer), lung (including small cell lung cancer, non-small cell lung cancer, large cell lung cancer and mesothelioma), endocrine system, bone, adrenal gland, thymus, liver, stomach, intestine (including gastric cancer), pancreas, bone marrow, hematological malignancies (such as lymphoma, leukemia, myeloma or lymphoid malignancies), bile duct, bladder, urinary tract, kidneys, skin, thyroid, head, neck, prostate and testis.

In some embodiments the neoplastic disease is a cancer selected from breast cancer (including triple negative breast cancer), gastric cancer, colorectal cancer, liver cancer (including hepatocellular cancer), endometrial cancer, ovarian cancer, esophageal cancer, lung cancer (including small cell lung cancer, non-small cell lung cancer), Kaposi's sarcoma, cervical cancer, pancreatic cancer, melanoma, prostate cancer, testicular cancer, cervical cancer, bladder cancer, head and neck cancer, brain tumor (e.g. glioma, medulloblastoma), neuroblastoma, retinoblastoma, Wilms' tumor, leukemia, e.g. acute myeloid leukemia (AML) (including Complex Karyotype AML) and malignant mesothelioma.

In some embodiments the neoplastic disease is breast cancer.

In some embodiments the neoplastic disease is triple negative breast cancer.

In some embodiments the neoplastic disease is ovarian cancer.

In some embodiments the neoplastic disease is gastric cancer.

In some embodiments the neoplastic disease is colorectal cancer.

In some embodiments the neoplastic disease is hepatocellular cancer.

In some embodiments the neoplastic disease is endometrial cancer (e.g. advanced or recurrent endometrial cancer).

In some embodiments the neoplastic disease is acute myeloid leukemia (AML) (including Complex Karyotype AML).

In some embodiments the neoplastic disease is lung cancer (e.g. small cell lung cancer, non-small cell lung cancer).

In some embodiments the neoplastic disease is cervical cancer (e.g. metastatic or recurrent cervical cancer).

In some embodiments the neoplastic disease is head and neck cancer (e.g. recurrent or metastatic squamous cell carcinoma of the head and neck).

In some embodiments the neoplastic disease is Wilms' tumor.

In some embodiments the neoplastic disease is a brain tumor (e.g. gliomas, such as progressive or recurrent gliomas, medulloblastoma, such as recurrent medul-loblastoma).

In some embodiments the neoplastic disease is neuroblastoma.

In some embodiments the neoplastic disease is testicular cancer (e.g. metastatic non-seminomatous germ cell tumor).

In some embodiments the neoplastic disease is bladder cancer (e.g. advanced bladder cancer, including those with abnormal renal function).

In some embodiments the neoplastic disease is retinoblastoma (e.g. recurrent or pro-gressive retinoblastoma.

In some embodiments the neoplastic disease is a cancer which is treatable with carboplatin. Such cancers include those listed in the carboplatin monograph, e.g. available at www.drugs.com/monograph/carboplatin.html. Such cancers include ovarian cancer (e.g. advanced epithelial ovarian cancer, recurrent ovarian cancer, early-stage ovarian cancer), lung cancer, (e.g. small call lung cancer, non-small cell lung cancer), cervical cancer (e.g. metastatic or recurrent cervical cancer), head and neck cancer (e.g. recurrent or metastatic squamous cell carcinoma of the head and neck, Wilms' Tumor, brain tumors (e.g. gliomas, such as progressive or recurrent gliomas, medulloblastoma, such as recurrent medulloblastoma), neuroblastoma, testicular cancer, bladder cancer, retinoblastoma, breast cancer (e.g. metastatic breast cancer), endometrial cancer e.g. advanced or recurrent endometrial cancer).

The cancer may be a primary tumor and/or metastases. The cancer may be derived from a solid or liquid (e.g. hematological or intraperitoneal) tumor. In some em-bodiments the neoplastic disease (e.g. cancer) to be treated is a tumor, e.g. a solid tumor.

### Administration

Administration of the pharmaceutical combinations of the invention includes admin-istration of the combination as a single formulation, as well as administration of the in-dividual agents of the combination as separate formulations. Preferably the compound of formula (I) or a pharmaceutically acceptable salt thereof and carboplatin are comprised in separate formulations. Preferably the compound of formula (I) or a pharmaceutically acceptable salt thereof and carboplatin are administered to a subject (preferably a human) intravenously.

In some embodiments, the combination of the invention is used for the treatment of cancer in a subject comprising administering to the subject a combination therapy, comprising a therapeutically effective amount of the compound of formula (I) or a pharmaceutically acceptable salt thereof and a therapeutically effective amount of carboplatin. These compounds are administered at therapeutically effective dosages, which when combined provide a beneficial effect e.g. as described herein. The skilled person will understand that therapeutically effective dosages for use in combination therapy may be lower than the dosages required to provide a therapeutic effect when using either agent as a monotherapy.

The administration of a pharmaceutical combination of the invention may result not only in a beneficial effect, e.g. with regard to alleviating, delaying progression of or in-hibiting the symptoms, but may also result in further beneficial effects, e.g. fewer side-effects, more durable therapeutic effect, an improved quality of life and/or a decreased morbidity, compared with a monotherapy applying only one of the pharmaceutically therapeutic agents used in the combination of the invention. It may also be the case that lower doses of the therapeutic agents of the combination of the invention can be used, for example, such that the dosages may not only often be smaller, but also may be applied less frequently, or can be used in order to diminish the incidence of side-effects observed with one of the combination partners alone.

The combinations of the present invention can be used in long-term therapy or as an adjuvant therapy in the context of other treatment strategies, as described above. Other possible treatments are therapy to maintain the subject's (e.g. patient's) status after tumor regression, or even preventive therapy, for example in subjects (e.g. patients) at risk.

The methods according to the invention may comprise (i) administration of the compound of formula (I) in free or pharmaceutically acceptable salt form and (ii) ad-ministration of carboplatin simultaneously, sequentially or separately in any order, in jointly therapeutically effective amounts, e.g. in synergistically effective amounts, e.g. in continuous or cyclic dosing schedules, e.g. corresponding to the amounts described herein. The individual combination partners of the combination of the invention may be administered separately at different times during the course of therapy or con-currently. The invention is therefore to be understood as embracing all such treatment regimens and the term “administering” is to be interpreted accordingly. Examples of treatment regimens for use with the invention are described in detail below.

The compound of formula (I) or a pharmaceutically acceptable salt thereof and carboplatin may be administered according to the same treatment schedule or may be administered according to independent treatment schedules. The treatment schedules may be cyclic or continuous.

A cyclic treatment schedule is defined by a repeated dosing schedule wherein the repeated element (a cycle) has a specific duration and wherein doses are administered on specific days within the cycle. A cycle may incorporate a period, usually at the end of the cycle, in which there is no administration (a “rest period”), e.g. to allow a period for recovery. A treatment cycle may be, e.g. 7 days, 14 days, 21 days, 28 days or longer. A continuous treatment schedule is a regular dosing schedule, which does not incorporate rest periods (i.e. periods that are longer than the regular interval between the doses). For example, doses may be administered once per day, twice per day, once every two days, once every three days etc. The treatment schedule, whether cyclic or continuous may be continued for as long as required (an “open-end treatment”) e.g. as long as the subject (e.g. patient) is receiving benefit judged by a physician overseeing the treatment.

When the compound of formula (I) or a pharmaceutically acceptable salt thereof and carboplatin are administered according to independent treatment schedules, the treatment schedules may both be cyclic, or one may be cyclic and the other may be continuous. When both treatment schedules are cyclic, the cycles of the two treatment schedules may be of the same duration or may be of different duration, and they may start on the same day or may start on different days. When the cycles are of the same duration the treatment cycles of both drugs will usually start on the same day.

In some embodiments the compound of formula (I) or a pharmaceutically acceptable salt thereof and carboplatin are administered according to cyclic treatment schedules which may be of the same duration, e.g. three weeks/21 days, with the respective treatment cycles usually starting on the same day (i.e. the same treatment cycle).

In some embodiments, the compound of formula (I) or a pharmaceutically acceptable salt thereof and carboplatin are administered (e.g. intravenously) according to a three week treatment cycle, wherein the compound of formula (I) or a pharmaceutically acceptable salt thereof is administered in week one of the treatment cycle, e.g. on day 1, followed by two rest weeks and carboplatin is administered in week one of the treatment cycle followed by two rest weeks, e.g. on day 1. Preferably the compound of formula (I) or a pharmaceutically acceptable salt thereof and carboplatin are administered to the subject according to the same three week treatment cycle (i.e. the treatment cycles start on the same day).

In some embodiments, the compound of formula (I) or a pharmaceutically acceptable salt thereof and carboplatin are administered (e.g. intravenously) according to a three week treatment cycle, wherein the compound of formula (I) or a pharmaceutically acceptable salt thereof is administered in week one and in week two of the treatment cycle, e.g. on day 1 and day 8, with week three being a rest week, and carboplatin is administered in week one of the treatment cycle followed by two rest weeks, e.g. on day 1. Preferably the compound of formula (I) or a pharmaceutically acceptable salt thereof and carboplatin are administered to the subject according to the same three week treatment cycle (i.e. the treatment cycles start on the same day).

Effective dosages of each of the combination partners employed in the combinations of the invention may vary depending on the pharmaceutical composition employed, the mode of administration, the condition being treated, and the severity of the condition being treated. Thus, the dosage regimen of the combination of the invention is selected in accordance with a variety of factors including the route of administration and the renal and hepatic function of the subject (e.g. patient).

The optimum ratios, individual and combined dosages, and concentrations of the combination partners of the pharmaceutical combination of the invention that yield efficacy without toxicity are based on the kinetics of the therapeutic agents' availability to target sites. They may be established using routine clinical testing and procedures that are well known in the art and will depend upon a variety of factors, such as the mode of administration, the condition being treated and the severity of the condition being treated, as well as the age, body weight, general health, gender and diet of the in-dividual and other medications the individual is taking. Likewise, frequency of dosage may vary depending on the compound used and the particular condition to be treated. Subjects (e.g. patients) may generally be monitored for therapeutic effectiveness using assays suitable for the condition being treated, which will be familiar to those of ordinary skill in the art.

In some embodiments the molar ratio of carboplatin to the compound of formula (I) or a pharmaceutically acceptable salt thereof per weekly dose in the combinations of the invention may be e.g. about 0.6:1 to about 250:1, e.g. about 0.6:1 to about 210:1, e.g. about 0.9:1 to about 250:1, e.g. about 0.9:1 to about 210:1, e.g. about 1.2:1 to about 10:1, e.g. about 1.2:1 to about 8:1, e.g. about 1.3:1 to about 8:1, e.g. about 1.3:1 to about 7:1, e.g. about 1.4:1 to about 32:1, e.g. about 1.4:1 to about 26:1, e.g. about 1.4:1 to about 7:1, e.g. about 1.4:1 to about 6:1, e.g. about 1.8:1 to about 16:1, e.g. about 1.8:1 to about 13:1, e.g. about 1.8:1 to about 9:1, e.g. about 1.8:1 to about 8:1, e.g. about 1.9:1 to about 8:1, e.g. about 1.9:1 to about 7:1, e.g. about 2.1:1 to about 32:1, e.g. about 2.1:1 to about 26:1, e.g. about 2.2:1 to about 14:1, e.g. about 2.2:1 to about 12:1, e.g. about 2.1:1 to about 7:1, e.g. about 2.1:1 to about 6:1, e.g. about 2.6:1 to about 16:1, e.g. about 2.6:1 to about 13:1, e.g. about 3.3:1 to about 14:1, e.g. about 3.3:1 to about 12:1. In some embodiments the molar ratio of carboplatin to the compound of formula (I) or a pharmaceutically acceptable salt thereof per weekly dose is e.g. about 2.1:1 to about 32:1.

In some embodiments the molar ratio of carboplatin to the compound of formula (I) or a pharmaceutically acceptable salt thereof per weekly dose may be e.g. up to about 250:1, e.g. up to about 210:1, e.g. up to about 32:1, e.g. up to about 26:1, e.g. up to about 16:1, e.g. up to about 14:1, e.g. up to about 13:1, e.g. up to about 12:1, e.g. up to about 9:1, e.g. up to about 8:1, e.g. up to about 7:1, e.g. up to about 6:1. In some em-bodiments the molar ratio of carboplatin to the compound of formula (I) or a pharmaceutically acceptable salt thereof per weekly dose is up to about 32:1.

In some embodiments the molar ratio of carboplatin to the compound of formula (I) or a pharmaceutically acceptable salt thereof per weekly dose may be e.g. at least about 0.6:1, e.g. at least about 0.9:1, e.g. at least about 1.2:1, e.g. at least about 1.3:1, e.g. at least about 1.4:1, e.g. at least about 1.8:1, e.g. at least about 1.9:1, e.g. at least about 2.1:1, e.g. at least about 2.2:1, e.g. at least about 2.6:1, e.g. at least about 3.3:1. In some embodiments the molar ratio of carboplatin to the compound of formula (I) or a pharmaceutically acceptable salt thereof per weekly dose is at least about 2.1:1.

The following examples of dosages are for humans. The doses of compound of formula (I) are given as mg per person irrespective of body weight or body surface area (BSA). Dosages of the compound of formula (I) as given below, including in Tables A, B and C refer to dosages of the free base. The dosages also apply to pharmaceutically acceptable salts of the compound of formula (I), except that when a pharmaceutically acceptable salt of the compound of formula (I) is used the stated mg dosage amount should be adjusted (i.e. increased) so that the molar amount of the pharmaceutically acceptable salt of the compound of formula (I) to be dosed is the same as the molar amount of the free base as given below. For example, a statement that the (human) weekly dosage amount of the compound of formula (I) is about 5 mg to about 480 mg in weeks when administered means that the compound of formula (I) or a pharmaceutically acceptable salt thereof is administered to a patient at a dose corre-sponding to the mole equivalent of about 5 mg to about 480 mg of the free base of the compound of formula (I) per week in weeks when administered. The dose per week in weeks when administered is the actual dose the patient receives in a given week, as opposed to the average weekly dose which may be less than the actual weekly dose when taking rest weeks into account.

In some embodiments the (human) weekly dosage amount of the compound of formula (I) is about 5 mg to about 480 mg in weeks when administered. In some em-bodiments the (human) weekly dosage amount of the compound of formula (I) is about 40 mg to about 200 mg in weeks when administered. In some embodiments the (human) weekly dosage amount of the compound of formula (I) is about 80 mg to about 160 mg in weeks when administered. In some embodiments the (human) weekly dosage amount of the compound of formula (I) is about 90 mg to about 130 mg in weeks when administered.

In some embodiments the (human) weekly dosage amount of the compound of formula (I) is about 140 mg to about 240 mg in weeks when administered. In some em-bodiments the (human) weekly dosage amount of the compound of formula (I) is about 160 mg to about 220 mg in weeks when administered. In some embodiments the (human) weekly dosage amount of the compound of formula (I) is about 180 mg to about 200 mg in weeks when administered.

Examples of weekly (human) dosage amounts of the compound of formula (I) in weeks when administered include about 10 mg to about 20 mg, about 20 mg to about 30 mg, about 30 mg to about 40 mg, about 40 mg to about 50 mg, about 50 mg to about 60 mg, about 60 mg to about 70 mg, about 70 mg to about 80 mg, about 80 mg to about 90 mg, about 90 mg to about 100 mg, about 100 mg to about 110 mg, about 110 mg to about 120 mg, about 120 mg to about 130 mg, about 130 mg to about 140 mg, about 140 mg to about 150 mg, about 150 mg to about 160 mg, about 160 mg to about 170 mg, about 170 mg to about 180 mg, about 180 mg to about 190 mg, about 190 mg to about 200 mg, about 200 mg to about 210 mg, about 210 mg to about 220 mg, about 220 mg to about 230 mg, about 230 mg to about 240 mg, about 240 mg to about 250 mg, about 250 mg to about 260 mg, about 260 mg to about 270 mg, about 270 mg to about 280 mg, about 280 mg to about 290 mg, about 290 mg to about 300 mg, about 300 mg to about 310 mg, about 310 mg to about 320 mg, about 320 mg to about 330 mg, about 330 mg to about 340 mg, about 340 mg to about 350 mg, about 350 mg to about 360 mg, about 360 mg to about 370 mg, about 370 mg to about 380 mg, about 380 mg to about 390 mg, about 390 mg to about 400 mg, about 400 mg to about 410 mg, about 410 mg to about 420 mg, about 420 mg to about 430 mg, about 430 mg to about 440 mg, about 440 mg to about 450 mg, about 450 mg to about 460 mg, about 460 mg to about 470 mg, and about 470 mg to about 480 mg.

Examples of specific (human) weekly dosage amounts of the compound of formula (I) in weeks when administered include about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, and about 480 mg.

The weekly dose of the compound of formula (I) or a pharmaceutically acceptable salt thereof may be administered in a single administration, e.g. substantially without any pause when administered intravenously. Alternatively the weekly dose may be administered in multiple administrations, e.g. in two or three administrations with pauses in between administrations when administered intravenously, e.g. of at least 30 minutes, e.g. at least one hour, e.g. at least two hours, e.g. at least 4 hours between ad-ministrations, e.g. 30 minutes to 12 hours, e.g. 30 minutes to 6 hours between administrations. Such multiple administrations may be on the same day or on separate days, e.g. on consecutive days or e.g. on the third day after the day of initial administration. Usually a weekly dose will be administered within a 24 hour period and will usually be substantially without any pause.

The compound of formula (I) or a pharmaceutically acceptable salt thereof is preferably administered intravenously. The duration of the infusion will usually be at least 30 minutes and may be up to 24 hours. In some embodiments the duration of the infusion is 30 minutes to 12 hours, e.g. 30 minutes to 6 hours, e.g. 30 minutes to 3 hours, e.g. one to two hours, e.g. about one hour.

Human dosage amounts of carboplatin are usually calculated according to the Calvert formula (Calvert et al., J. Clin. Oncol. 1989; 7(11):1748-56). The Calvert formula in-corporates the glomerular filtration rate as its key variable. The most common way of estimating the glomerular filtration rate for the purposes of the Calvert formula is by way of the creatine clearance calculated according to the Cockroft-Gault equation (Cockcroft, Gault, Nephron 1976, 16:31-41; Ainsworth et al. Annals of Oncology 2012, 23:1845-1853; Bjornsson, Clin Pharmacokinet. 1979, 4(3):200-222). Therefore for the purposes of the present invention, the carboplatin dose is calculated according to the following formula:

Carboplatin dose [mg]=target AUC [mg/mL×min]×(CL_{CR}[mL/min]+25)

Where CL_{CR} is the creatine clearance calculated according to the Cockroft-Gault equation.

For example, an AUC5 dose is calculated as follows:

Carboplatin dose [mg]=5×(CL_{CR}[mL/min]+25)

The maximum dose of carboplatin (this dose should not be exceeded) is target AUC (mg/mL/min)×(150):
For a target AUC6, the maximum dose is 6×150=900 mg
For a target AUC5, the maximum dose is 5×150=750 mg
For a target AUC4, the maximum dose is 4×150=600 mg

In some embodiments carboplatin is administered according to AU4 per week in weeks when administered. In some embodiments carboplatin is administered according to AU5 per week in weeks when administered. In some embodiments carboplatin is administered according to AU6 per week in weeks when administered.

The weekly dose of carboplatin may be administered in a single administration, e.g. without pause when administered intravenously. Alternatively the weekly dose may be administered in multiple administrations, e.g. in two or three administrations with pauses in between administrations when administered intravenously, e.g. of at least about 30 minutes, e.g. at least about one hour, e.g. at least about two hours, e.g. at least about 4 hours between administrations, e.g. about 30 minutes to about 12 hours, e.g. about 30 minutes to about 6 hours. Such multiple administrations may be on the same day or on separate days, e.g. on consecutive days or on the third day after the initial ad-ministration. Usually a weekly dose will be administered within a 24 hour period and will usually be substantially without any pause.

Carboplatin is preferably administered intravenously. The duration of the infusion will usually be at least about 30 minutes and may be up to about 24 hours. In some em-bodiments the duration of the infusion is about 30 minutes to about 12 hours, e.g. about 30 minutes to about 6 hours, e.g. about 30 minutes to about 3 hours, e.g. about one to about two hours, e.g. about 30 minutes.

In some embodiments the (human) weekly dosage amounts of the compound of formula (I) in weeks when administered and the (human) weekly dosage amount of carboplatin in weeks when administered is as indicated in any one of embodiments 1A to 21A in Table A.

TABLE A

| Embodiment | Carboplatin | Compound of formula (I) (mg) |
|---|---|---|
| 1A | AUC4 | 5-480 |
| 2A | AUC4 | 40-200 |
| 3A | AUC4 | 80-160 |
| 4A | AUC4 | 90-130 |
| 5A | AUC4 | 140-240 |
| 6A | AUC4 | 160-220 |
| 7A | AUC4 | 180-200 |
| 8A | AUC5 | 5-480 |
| 9A | AUC5 | 40-200 |
| 10A | AUC5 | 80-160 |
| 11A | AUC5 | 90-130 |
| 12A | AUC5 | 140-240 |
| 13A | AUC5 | 160-220 |
| 14A | AUC5 | 180-200 |
| 15A | AUC6 | 5-480 |
| 16A | AUC6 | 40-200 |
| 17A | AUC6 | 80-160 |
| 18A | AUC6 | 90-130 |
| 19A | AUC6 | 140-240 |
| 20A | AUC6 | 160-220 |
| 21A | AUC6 | 180-200 |

As per the statement above, embodiment 1A refers to the situation wherein carboplatin is administered to a patient at AUC4 per week in weeks when administered and the compound of formula (I) or a pharmaceutically acceptable salt thereof is administered to a patient at a dose corresponding to the mole equivalent of about 5 mg to about 480 mg of the free base of the compound of formula (I) per week in weeks when administered. The same applies analogously to embodiments 2A to 21A.

In some embodiments the (human) weekly dosage amounts of the compound of formula (I) in weeks when administered and the (human) weekly dosage amount of carboplatin in weeks when administered is as indicated in any one of embodiments 1B to 144B in Table B.

TABLE B

| | | Compound |
|---|---|---|
| | | of formula |
| Embodiment | Carboplatin | (I) |
| 1B | AUC4 | 5-10 |
| 2B | AUC4 | 10-20 |
| 3B | AUC4 | 20-30 |
| 4B | AUC4 | 30-40 |
| 5B | AUC4 | 40-50 |
| 6B | AUC4 | 50-60 |
| 7B | AUC4 | 60-70 |
| 8B | AUC4 | 70-80 |
| 9B | AUC4 | 80-90 |
| 10B | AUC4 | 90-100 |
| 11B | AUC4 | 100-110 |
| 12B | AUC4 | 110-120 |
| 13B | AUC4 | 120-130 |
| 14B | AUC4 | 130-140 |
| 15B | AUC4 | 140-150 |
| 16B | AUC4 | 150-160 |
| 17B | AUC4 | 160-170 |
| 18B | AUC4 | 170-180 |
| 19B | AUC4 | 180-190 |
| 20B | AUC4 | 190-200 |
| 21B | AUC4 | 200-210 |
| 22B | AUC4 | 210-220 |
| 23B | AUC4 | 220-230 |
| 24B | AUC4 | 230-240 |
| 25B | AUC4 | 240-250 |
| 26B | AUC4 | 250-260 |
| 27B | AUC4 | 260-270 |
| 28B | AUC4 | 270-280 |
| 29B | AUC4 | 280-290 |
| 30B | AUC4 | 290-300 |
| 31B | AUC4 | 300-310 |
| 32B | AUC4 | 310-320 |
| 33B | AUC4 | 320-330 |
| 34B | AUC4 | 330-340 |
| 35B | AUC4 | 340-350 |
| 36B | AUC4 | 350-360 |
| 37B | AUC4 | 360-370 |
| 38B | AUC4 | 370-380 |
| 39B | AUC4 | 380-390 |
| 40B | AUC4 | 390-400 |
| 41B | AUC4 | 400-410 |
| 42B | AUC4 | 410-420 |
| 43B | AUC4 | 420-430 |
| 44B | AUC4 | 430-440 |
| 45B | AUC4 | 440-450 |
| 46B | AUC4 | 450-460 |
| 47B | AUC4 | 460-470 |
| 48B | AUC4 | 470-480 |
| 49B | AUC5 | 5-15 |
| 50B | AUC5 | 10-20 |
| 51B | AUC5 | 20-30 |
| 52B | AUC5 | 30-40 |
| 53B | AUC5 | 40-50 |
| 54B | AUC5 | 50-60 |
| 55B | AUC5 | 60-70 |
| 56B | AUC5 | 70-80 |
| 57B | AUC5 | 80-90 |
| 58B | AUC5 | 90-100 |
| 59B | AUC5 | 100-110 |
| 60B | AUC5 | 110-120 |
| 61B | AUC5 | 120-130 |
| 62B | AUC5 | 130-140 |
| 63B | AUC5 | 140-150 |
| 64B | AUC5 | 150-160 |
| 65B | AUC5 | 160-170 |
| 66B | AUC5 | 170-180 |
| 67B | AUC5 | 180-190 |
| 68B | AUC5 | 190-200 |
| 69B | AUC5 | 200-210 |
| 70B | AUC5 | 210-220 |
| 71B | AUC5 | 220-230 |
| 72B | AUC5 | 230-240 |
| 73B | AUC5 | 240-250 |
| 74B | AUC5 | 250-260 |
| 75B | AUC5 | 260-270 |
| 76B | AUC5 | 270-280 |
| 77B | AUC5 | 280-290 |
| 78B | AUC5 | 290-300 |
| 79B | AUC5 | 300-310 |
| 80B | AUC5 | 310-320 |
| 81B | AUC5 | 320-330 |
| 82B | AUC5 | 330-340 |
| 83B | AUC5 | 340-350 |
| 84B | AUC5 | 350-360 |
| 85B | AUC5 | 360-370 |
| 86B | AUC5 | 370-380 |
| 87B | AUC5 | 380-390 |
| 88B | AUC5 | 390-400 |
| 89B | AUC5 | 400-410 |
| 90B | AUC5 | 410-420 |
| 91B | AUC5 | 420-430 |
| 92B | AUC5 | 430-440 |
| 93B | AUC5 | 440-450 |
| 94B | AUC5 | 450-460 |
| 95B | AUC5 | 460-470 |
| 96B | AUC5 | 470-480 |
| 97B | AUC6 | 5-10 |
| 98B | AUC6 | 10-20 |
| 99B | AUC6 | 20-30 |
| 100B | AUC6 | 30-40 |
| 101B | AUC6 | 40-50 |
| 102B | AUC6 | 50-60 |
| 103B | AUC6 | 60-70 |
| 104B | AUC6 | 70-80 |
| 105B | AUC6 | 80-90 |
| 106B | AUC6 | 90-100 |
| 107B | AUC6 | 100-110 |
| 108B | AUC6 | 110-120 |
| 109B | AUC6 | 120-130 |
| 110B | AUC6 | 130-140 |
| 111B | AUC6 | 140-150 |
| 112B | AUC6 | 150-160 |
| 113B | AUC6 | 160-170 |
| 114B | AUC6 | 170-180 |
| 115B | AUC6 | 180-190 |
| 116B | AUC6 | 190-200 |
| 117B | AUC6 | 200-210 |
| 118B | AUC6 | 210-220 |
| 119B | AUC6 | 220-230 |
| 120B | AUC6 | 230-240 |
| 121B | AUC6 | 240-250 |
| 122B | AUC6 | 250-260 |
| 123B | AUC6 | 260-270 |
| 124B | AUC6 | 270-280 |
| 125B | AUC6 | 280-290 |
| 126B | AUC6 | 290-300 |
| 127B | AUC6 | 300-310 |
| 128B | AUC6 | 310-320 |
| 129B | AUC6 | 320-330 |
| 130B | AUC6 | 330-340 |
| 131B | AUC6 | 340-350 |
| 132B | AUC6 | 350-360 |
| 133B | AUC6 | 360-370 |
| 134B | AUC6 | 370-380 |
| 135B | AUC6 | 380-390 |
| 136B | AUC6 | 390-400 |
| 137B | AUC6 | 400-410 |
| 138B | AUC6 | 410-420 |
| 139B | AUC6 | 420-430 |
| 140B | AUC6 | 430-440 |
| 141B | AUC6 | 440-450 |
| 142B | AUC6 | 450-460 |
| 143B | AUC6 | 460-470 |
| 144B | AUC6 | 470-480 |

As per the statement above, embodiment 1B refers to the situation wherein carboplatin is administered to a patient at AUC4 per week in weeks when administered and the compound of formula (I) or a pharmaceutically acceptable salt thereof is administered to a patient at a dose corresponding to the mole equivalent of about 5 mg to about 10 mg of the free base of the compound of formula (I) per week in weeks when administered. The same applies analogously to embodiments 2B to 144B.

In some embodiments the treatment cycle duration of the compound of formula (I) or a pharmaceutically acceptable salt thereof and of carboplatin is three weeks and the (human) weekly dosage of the compound of formula (I) and carboplatin in weeks when administered and the week(s) of administration within the cycle are as indicated in any one of embodiments 1C to 42C in Table C, wherein the compound of formula (I) and carboplatin are administered according to the same treatment cycle (i.e. starting on the same day).

TABLE C

| | Cycle | Carboplatin | | Compound of formula (I) | |
|---|---|---|---|---|---|
| | duration | Weeks of | | Weeks of | Weekly |
| Embodiment | (weeks) | administration | Weekly dosage | administration | dosage (mg) |
| 1C | 3 | 1 | AUC4 | 1 | 5-480 |
| 2C | 3 | 1 | AUC4 | 1 | 40-200 |
| 3C | 3 | 1 | AUC4 | 1 | 80-160 |
| 4C | 3 | 1 | AUC4 | 1 | 90-130 |
| 5C | 3 | 1 | AUC4 | 1 | 140-240 |
| 6C | 3 | 1 | AUC4 | 1 | 160-220 |
| 7C | 3 | 1 | AUC4 | 1 | 180-200 |
| 8C | 3 | 1 | AUC4 | 1, 2 | 5-480 |
| 9C | 3 | 1 | AUC4 | 1, 2 | 40-200 |
| 10C | 3 | 1 | AUC4 | 1, 2 | 80-160 |
| 11C | 3 | 1 | AUC4 | 1, 2 | 90-130 |
| 12C | 3 | 1 | AUC4 | 1, 2 | 140-240 |
| 13C | 3 | 1 | AUC4 | 1, 2 | 160-220 |
| 14C | 3 | 1 | AUC4 | 1, 2 | 180-200 |
| 15C | 3 | 1 | AUC5 | 1 | 5-480 |
| 16C | 3 | 1 | AUC5 | 1 | 40-200 |
| 17C | 3 | 1 | AUC5 | 1 | 80-160 |
| 18C | 3 | 1 | AUC5 | 1 | 90-130 |
| 19C | 3 | 1 | AUC5 | 1 | 140-240 |
| 20C | 3 | 1 | AUC5 | 1 | 160-220 |
| 21C | 3 | 1 | AUC5 | 1 | 180-200 |
| 22C | 3 | 1 | AUC5 | 1, 2 | 5-480 |
| 23C | 3 | 1 | AUC5 | 1, 2 | 40-200 |
| 24C | 3 | 1 | AUC5 | 1, 2 | 80-160 |
| 25C | 3 | 1 | AUC5 | 1, 2 | 90-130 |
| 26C | 3 | 1 | AUC5 | 1, 2 | 140-240 |
| 27C | 3 | 1 | AUC5 | 1, 2 | 160-220 |
| 28C | 3 | 1 | AUC5 | 1, 2 | 180-200 |
| 29C | 3 | 1 | AUC6 | 1 | 5-480 |
| 30C | 3 | 1 | AUC6 | 1 | 40-200 |
| 31C | 3 | 1 | AUC6 | 1 | 80-160 |
| 32C | 3 | 1 | AUC6 | 1 | 90-130 |
| 33C | 3 | 1 | AUC6 | 1 | 140-240 |
| 34C | 3 | 1 | AUC6 | 1 | 160-220 |
| 35C | 3 | 1 | AUC6 | 1 | 180-200 |
| 36C | 3 | 1 | AUC6 | 1, 2 | 5-480 |
| 37C | 3 | 1 | AUC6 | 1, 2 | 40-200 |
| 38C | 3 | 1 | AUC6 | 1, 2 | 80-160 |
| 39C | 3 | 1 | AUC6 | 1, 2 | 90-130 |
| 40C | 3 | 1 | AUC6 | 1, 2 | 140-240 |
| 41C | 3 | 1 | AUC6 | 1, 2 | 160-220 |
| 42C | 3 | 1 | AUC6 | 1, 2 | 180-200 |

As per the statement above, embodiment 1C refers to the situation wherein the compound of formula (I) or a pharmaceutically acceptable salt thereof and carboplatin are administered (e.g. intravenously) to a patient according to the same three week treatment cycle, wherein the compound of formula (I) or a pharmaceutically acceptable salt thereof is administered in week one of the treatment cycle followed by two rest weeks and carboplatin is administered in week one of the treatment cycle followed by two rest week, and wherein carboplatin is administered to a patient at AUC4 per week in weeks when administered and the compound of formula (I) or a pharmaceutically acceptable salt thereof is administered to a patient at a dose corresponding to the mole equivalent of about 5 mg to about 480 mg of the free base of the compound of formula (I) per week in weeks when administered. The same applies analogously to em-bodiments 2C to 42C.

In some embodiments the treatment cycle duration of the compound of formula (I) or a pharmaceutically acceptable salt thereof and carboplatin is 21 days and the (human) weekly dosage of the compound of formula (I) and carboplatin in weeks when administered and the days of administration within the cycle are as indicated in any one of embodiments 1D to 42D in Table D, wherein the compound of formula (I) and carboplatin are administered according to the same treatment cycle (i.e. starting on the same day).

TABLE D

| | Cycle | Carboplatin | | Compound of formula (I) | |
|---|---|---|---|---|---|
| | duration | Days of | | Days of | Weekly |
| Embodiment | (days) | administration | Weekly dosage | administration | dosage (mg) |
| 1D | 21 | 1 | AUC4 | 1 | 5-480 |
| 2D | 21 | 1 | AUC4 | 1 | 40-200 |
| 3D | 21 | 1 | AUC4 | 1 | 80-160 |
| 4D | 21 | 1 | AUC4 | 1 | 90-130 |
| 5D | 21 | 1 | AUC4 | 1 | 140-240 |
| 6D | 21 | 1 | AUC4 | 1 | 160-220 |
| 7D | 21 | 1 | AUC4 | 1 | 180-200 |
| 8D | 21 | 1 | AUC4 | 1, 8 | 5-480 |
| 9D | 21 | 1 | AUC4 | 1, 8 | 40-200 |
| 10D | 21 | 1 | AUC4 | 1, 8 | 80-160 |
| 11D | 21 | 1 | AUC4 | 1, 8 | 90-130 |
| 12D | 21 | 1 | AUC4 | 1, 8 | 140-240 |
| 13D | 21 | 1 | AUC4 | 1, 8 | 160-220 |
| 14D | 21 | 1 | AUC4 | 1, 8 | 180-200 |
| 15D | 21 | 1 | AUC5 | 1 | 5-480 |
| 16D | 21 | 1 | AUC5 | 1 | 40-200 |
| 17D | 21 | 1 | AUC5 | 1 | 80-160 |
| 18D | 21 | 1 | AUC5 | 1 | 90-130 |
| 19D | 21 | 1 | AUC5 | 1 | 140-240 |
| 20D | 21 | 1 | AUC5 | 1 | 160-220 |
| 21D | 21 | 1 | AUC5 | 1 | 180-200 |
| 22D | 21 | 1 | AUC5 | 1, 8 | 5-480 |
| 23D | 21 | 1 | AUC5 | 1, 8 | 40-200 |
| 24D | 21 | 1 | AUC5 | 1, 8 | 80-160 |
| 25D | 21 | 1 | AUC5 | 1, 8 | 90-130 |
| 26D | 21 | 1 | AUC5 | 1, 8 | 140-240 |
| 27D | 21 | 1 | AUC5 | 1, 8 | 160-220 |
| 28D | 21 | 1 | AUC5 | 1, 8 | 180-200 |
| 29D | 21 | 1 | AUC6 | 1 | 5-480 |
| 30D | 21 | 1 | AUC6 | 1 | 40-200 |
| 31D | 21 | 1 | AUC6 | 1 | 80-160 |
| 32D | 21 | 1 | AUC6 | 1 | 90-130 |
| 33D | 21 | 1 | AUC6 | 1 | 140-240 |
| 34D | 21 | 1 | AUC6 | 1 | 160-220 |
| 35D | 21 | 1 | AUC6 | 1 | 180-200 |
| 36D | 21 | 1 | AUC6 | 1 ,8 | 5-480 |
| 37D | 21 | 1 | AUC6 | 1, 8 | 40-200 |
| 38D | 21 | 1 | AUC6 | 1, 8 | 80-160 |
| 39D | 21 | 1 | AUC6 | 1, 8 | 90-130 |
| 40D | 21 | 1 | AUC6 | 1, 8 | 140-240 |
| 41D | 21 | 1 | AUC6 | 1, 8 | 160-220 |
| 42D | 21 | 1 | AUC6 | 1, 8 | 180-200 |

As per the statement above, embodiment 1D refers to the situation wherein the compound of formula (I) or a pharmaceutically acceptable salt thereof and carboplatin are administered (e.g. intravenously) to a patient according to the same 21 day treatment cycle, wherein the compound of formula (I) or a pharmaceutically acceptable salt thereof is administered on day 1 of the treatment cycle and carboplatin is administered on day 1 of the treatment cycle, and wherein carboplatin is administered to a patient at AUC4 per week in weeks when administered and the compound of formula (I) or a pharmaceutically acceptable salt thereof is administered to a patient at a dose corresponding to the mole equivalent of about 5 mg to about 480 mg of the free base of the compound of formula (I) per week in weeks when administered. The same applies analogously to embodiments 2D to 42D.

In some embodiments the compound of formula (I) may be administered twice per week in weeks when administered.

As shown in the Examples below, it can be advantageous to administer the dose of carboplatin prior to the dose of the compound of formula (I) or pharmaceutically acceptable salt thereof, in particular when carboplatin and the compound of formula (I) or pharmaceutically acceptable salt thereof are administered at the same time, e.g. on the same day.

Accordingly, in some embodiments carboplatin is administered to the subject before the compound of formula (I) or a pharmaceutically acceptable salt thereof is administered to the subject. In some embodiments when carboplatin and the compound of formula (I) or a pharmaceutically acceptable salt thereof are both administered to the subject within a 48 hour period carboplatin is administered to the subject before the compound of formula (I) or a pharmaceutically acceptable salt thereof is administered to the subject. In some embodiments when carboplatin and the compound of formula (I) or a pharmaceutically acceptable salt thereof are both administered to the subject within a 24 hour period then carboplatin is administered to the subject before the compound of formula (I) or a pharmaceutically acceptable salt thereof is administered to the subject

There may be a pause between administration of carboplatin (first) and the compound of formula (I) or a pharmaceutically acceptable salt thereof (second), e.g. of at least about 30 minutes, e.g. at least about one hour, e.g. at least about 2 hours, e.g. at least about 4 hours, e.g. at least about 24 hours, e.g. up to about 4 hours, e.g. up to about 6 hours, e.g. up to about 12 hours, e.g. up to about 24 hours, e.g. about 30 minutes to about 24 hours, e.g. about 30 minutes to about 12 hours, e.g. about 30 minutes to about 6 hours, e.g. about 2 to about 4 hours, e.g. about 4 to about 6 hours between administrations.

Accordingly, in some embodiments carboplatin is administered to the subject at least about 30 minutes before the compound of formula (I) or pharmaceutically acceptable salt thereof is administered to the subject. In some embodiments carboplatin is administered to the subject at least about 1 hour before the compound of formula (I) or pharmaceutically acceptable salt thereof is administered to the subject. In some em-bodiments carboplatin is administered to the subject at least about 4 hours before the compound of formula (I) or pharmaceutically acceptable salt thereof is administered to the subject. In some embodiments carboplatin is administered to the subject at least about 24 hours before the compound of formula (I) or pharmaceutically acceptable salt thereof is administered to the subject.

In some embodiments when carboplatin and the compound of formula (I) or a pharmaceutically acceptable salt thereof are both administered to the subject within a 24 hour period then carboplatin is administered to the subject at least about 30 minutes before the compound of formula (I) or a pharmaceutically acceptable salt thereof is administered to the subject. In some embodiments when carboplatin and the compound of formula (I) or a pharmaceutically acceptable salt thereof are both administered to the subject within a 24 hour period then carboplatin is administered to the subject at least about 1 hour before (the scheduled dose of) the compound of formula (I) or a pharmaceutically acceptable salt thereof is administered to the subject. In some em-bodiments when carboplatin and the compound of formula (I) or a pharmaceutically acceptable salt thereof are both administered to the subject within a 24 hour period carboplatin is administered to the subject at least 4 hours before the compound of formula (I) or a pharmaceutically acceptable salt thereof is administered to the subject. In some embodiments when carboplatin and the compound of formula (I) or a pharmaceutically acceptable salt thereof are both administered to the subject within a 48 hour period then carboplatin is administered to the subject at least about 24 hours before the compound of formula (I) or a pharmaceutically acceptable salt thereof is administered to the subject.

In some embodiments when carboplatin and the compound of formula (I) or a pharmaceutically acceptable salt thereof are both administered to the subject within a 48 hour period then carboplatin is administered to the subject up to 24 hours before the compound of formula (I) or a pharmaceutically acceptable salt thereof is administered to the subject. In some embodiments when carboplatin and the compound of formula (I) or a pharmaceutically acceptable salt thereof are both administered to the subject within a 24 hour period then carboplatin is administered to the subject up to 12 hours before the compound of formula (I) or a pharmaceutically acceptable salt thereof is administered to the subject. In some embodiments when carboplatin and the compound of formula (I) or a pharmaceutically acceptable salt thereof are both administered to the subject within a 24 hour period then carboplatin is administered to the subject up to 6 hours before the compound of formula (I) or a pharmaceutically acceptable salt thereof is administered to the subject. In some embodiments when carboplatin and the compound of formula (I) or a pharmaceutically acceptable salt thereof are both administered to the subject within a 24 hour period carboplatin is administered to the subject up to 4 hours before the compound of formula (I) or a pharmaceutically acceptable salt thereof is administered to the subject. In some embodiments when carboplatin and the compound of formula (I) or a pharmaceutically acceptable salt thereof are both administered to the subject within a 24 hour period then carboplatin is administered to the subject up to one hour before the compound of formula (I) or a pharmaceutically acceptable salt thereof is administered to the subject.

In some embodiments when carboplatin and the compound of formula (I) or a pharmaceutically acceptable salt thereof are both administered to the subject within a 48 hour period carboplatin is administered to the subject about 30 minutes to about 24 hours before the compound of formula (I) or a pharmaceutically acceptable salt thereof is administered to the subject. In some embodiments when carboplatin and the compound of formula (I) or a pharmaceutically acceptable salt thereof are both administered to the subject within a 24 hour period then carboplatin is administered to the subject about 30 minutes to about 12 hours before the compound of formula (I) or a pharmaceutically acceptable salt thereof is administered to the subject. In some em-bodiments when carboplatin and the compound of formula (I) or a pharmaceutically acceptable salt thereof are both administered to the subject within a 24 hour period then carboplatin is administered to the subject about 30 minutes to about 6 hours before the compound of formula (I) or a pharmaceutically acceptable salt thereof is administered to the subject. In some embodiments when carboplatin and the compound of formula (I) or a pharmaceutically acceptable salt thereof are both administered to the subject within a 24 hour period then carboplatin is administered to the subject about 2 to about 4 hours before the compound of formula (I) or a pharmaceutically acceptable salt thereof is administered to the subject. In some embodiments when carboplatin and the compound of formula (I) or a pharmaceutically acceptable salt thereof are both administered to the subject within a 24 hour period then carboplatin is administered to the subject about 4 to about 6 hours before the compound of formula (I) or a pharmaceutically acceptable salt thereof is administered to the subject.

In some embodiments carboplatin is administered to the subject before the compound of formula (I) or a pharmaceutically acceptable salt thereof is administered to the subject, and wherein the neoplastic disease to be treated is breast cancer, e.g. triple negative breast cancer, or ovarian cancer.

In some embodiments when carboplatin and the compound of formula (I) or a pharmaceutically acceptable salt thereof are both administered to the subject within a 48 hour period then carboplatin is administered to the subject up to 24 hours before the compound of formula (I) or a pharmaceutically acceptable salt thereof is administered to the subject and wherein the neoplastic disease to be treated is breast cancer, e.g. triple negative breast cancer, or ovarian cancer.

In some embodiments when carboplatin and the compound of formula (I) or a pharmaceutically acceptable salt thereof are both administered to the subject within a 24 hour period then carboplatin is administered to the subject up to 6 hours before the compound of formula (I) or a pharmaceutically acceptable salt thereof is administered to the subject, and wherein the neoplastic disease to be treated is breast cancer, e.g. triple negative breast cancer, or ovarian cancer.

In some embodiments when carboplatin and the compound of formula (I) or a pharmaceutically acceptable salt thereof are both administered to the subject within a 24 hour period then carboplatin is administered to the subject up to 4 hours before the compound of formula (I) or a pharmaceutically acceptable salt thereof is administered to the subject, and wherein the neoplastic disease to be treated is breast cancer, e.g. triple negative breast cancer, or ovarian cancer.

In some embodiments when carboplatin and the compound of formula (I) or a pharmaceutically acceptable salt thereof are both administered to the subject within a 48 hour period then carboplatin is administered to the subject about 30 minutes to about 24 hours before the compound of formula (I) or a pharmaceutically acceptable salt thereof is administered to the subject, and wherein the neoplastic disease to be treated is breast cancer, e.g. triple negative breast cancer, or ovarian cancer.

The potential for administration of carboplatin prior to the compound of formula (I) or a pharmaceutically acceptable salt thereof as described above applies in particular to the embodiments described in Tables A, B, C and D.

Generally, the compound of formula (I) or a pharmaceutically acceptable salt thereof and carboplatin will be administered at dosages that do not exceed the maximum tolerated dose (MTD) for a particular mode of administration and indication, as determined in a clinical dose escalation study.

### Formulations

The compound of formula (I) or a pharmaceutically acceptable salt thereof and carboplatin may be provided as a combined medicament, but each will usually be provided as a single-agent medicaments. Separate pharmaceutical compositions have a number of advantages, for example, to allow different dosing schedules, different dosages and/or different routes of administration for each compound. When provided as single-agent medicaments the combination may be for separate, simultaneous or sequential administration.

The compounds of the invention may be formulated as pharmaceutical compositions for non-parenteral administration, such as nasal, buccal, rectal, pulmonary, vaginal, sublingual, topical, transdermal, ophthalmic, otic or, especially, for oral administration, e.g. in the form of oral solid dosage forms, e.g. granules, pellets, powders, tablets, film or sugar coated tablets, effervescent tablets, hard and soft gelatin or HPMC capsules, coated as applicable, orally disintegrating tablets, oral solutions, lipid emulsions or suspensions, or for parenteral administration, such as intravenous, intramuscular, or sub-cutaneous, intrathecal, intradermal or epidural administration, to mammals, especially humans, e.g. in the form of solutions, lipid emulsions or suspensions containing mi-croparticles or nanoparticles. Liposomal formulations provide a convenient way of co-formulating compounds with different solubility (see e.g. Bulbake et al. Pharmaceutics 2017, 9(2):12). The compositions may comprise the active ingredient(s) alone or, preferably, together with a pharmaceutically acceptable carrier.

The pharmaceutical compositions can be processed with pharmaceutically inert, inorganic or organic excipients for the production of oral solid dosage forms, e.g. granules, pellets, powders, tablets, film or sugar coated tablets, effervescent tablets, hard gelatin or HPMC capsules or orally disintegrating tablets. Fillers e.g. lactose, cellulose, mannitol, sorbitol, calcium phosphate, starch or derivatives thereof, binders e.g. cellulose, starch, polyvinylpyrrolidone, or derivatives thereof, glidants e.g. talcum, stearic acid or its salts, flowing agents e.g. fumed silica, can be used as such excipients for formulating and manufacturing of oral solid dosage forms, such as granules, pellets, powders, tablets, film or sugar coated tablets, effervescent tablets, hard gelatin or HPMC capsules, or orally disintegrating tablets. Suitable excipients for soft gelatin capsules are e.g. vegetable oils, waxes, fats, semisolid and liquid polyols etc.

Suitable excipients for the manufacture of oral solutions, lipid emulsions or suspensions are e.g. water, alcohols, polyols, saccharose, invert sugar, glucose etc. Suitable excipients for parenteral formulations are e.g. water, alcohols, polyols, glycerol, vegetable oils, lecithin, surfactants etc. Moreover, the pharmaceutical preparations can contain preservatives, solubilizers, stabilizers, wetting agents, emulsifiers, sweeteners, colorants, flavorants, salts for varying the osmotic pressure, buffers, masking agents or antioxidants. They can also contain other therapeutically valuable substances.

Pharmaceutical compositions suitable for injectable use include sterile aqueous solutions (where water soluble) or dispersions and sterile powders for the extem-poraneous preparation of sterile injectable solutions or dispersion. For intravenous ad-ministration, suitable carriers include physiological saline, bacteriostatic water, Cremophor® EL or phosphate buffered saline (PBS). The carrier can be a solvent or dispersion medium containing, for example, water, ethanol, polyol (for example, glycerol, propylene glycol, and liquid polyethylene glycol, and the like), and suitable mixtures thereof. For intravenous injection of strongly lipophilic molecules it can be advantageous to include solubilizers in the formulation, for example surfactants, polymeric surfactants, polymers, complexing agents and/or co-solvents, which may significantly increase the solubility of the compounds in water. Examples of solubilizers include polyethylene glycol, propylene glycol, ethanol, glycerol and cyclodextrins (e.g. sulfobutyl ether-β-cyclodextrins).

In some embodiments the compound of formula (I) as the free base is provided as a pharmaceutical composition comprising a β-cyclodextrin e.g. for intravenous admin-istration. The β-cyclodextrin may be sulfobutyl ether-β-cyclodextrin, e.g. CAS 182410-00-0, such as Captisol™ (Ligand) or Dexolve™ (Cyclolab).

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

In addition pharmaceutical compositions used in the invention optionally include buffers such as phosphate, citrate, or other organic acids; antioxidants including butylated hydroxytoluene (BHT), butylated hydroxyanisole (BHA), ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagines, arginine or lysine; monosaccharides, disaccharides, or other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™, or PEG.

Optionally, the pharmaceutical compositions contain a pharmaceutically acceptable preservative. In some embodiments the preservative concentration ranges from 0.1 to 2.0 percent, typically v/v. Suitable preservatives include those known in the pharmaceutical arts, such as benzyl alcohol, phenol, m-cresol, methylparaben, and propy-lparaben.

In some embodiments the compound of formula (I) or a pharmaceutically acceptable salt thereof is formulated for intravenous administration with a suitable acceptable carrier. In some embodiments carboplatin is formulated for intravenous administration with a suitable acceptable carrier.

### Kits

The invention also provides pharmaceutical products such as kits which may include a container with the compound of formula (I) or a pharmaceutically acceptable salt thereof and carboplatin, which can be provided in amounts sufficient for admin-istration, e.g. in pharmaceutically acceptable amounts. The kits can thus include multiple containers that each include pharmaceutically effective amounts of the active ingredients. Optionally, instruments and/or devices necessary for administering the pharmaceutical composition(s) can also be included in the kits. Furthermore, the kits can include additional components, such as instructions or administration schedules, for treating a patient with a neoplastic disease with the combinations of the invention.

Accordingly, in a further aspect the invention provides a pharmaceutical product such as a kit for use in treating a neoplastic disease e.g. as described herein, wherein the compound of formula (I) or a pharmaceutically acceptable salt thereof and carboplatin are provided as single-agent medicaments. In some embodiments, the kit further comprises instructions for simultaneous, separate or sequential administration thereof for use in the treatment of a neoplastic disease.

### Paragraphs

The following numbered paragraphs describe particular embodiments of the invention.
Paragraph 1. A pharmaceutical combination comprising (a) a compound of formula (I) or a pharmaceutically acceptable salt thereof; and (b) carboplatin.
Paragraph 2. The pharmaceutical combination according to Paragraph 1, wherein the compound of formula (I) or a pharmaceutically acceptable salt thereof and carboplatin are comprised in separate formulations, each formulation preferably for intravenous administration.
Paragraph 3. The pharmaceutical combination according to Paragraph 1 or Paragraph 2, wherein the molar ratio of carboplatin to the compound of formula (I) or a pharmaceutically acceptable salt thereof per weekly dose is about 0.6:1 to about 250:1.
Paragraph 4. The pharmaceutical combination according to Paragraph 1 or Paragraph 2, wherein the molar ratio of carboplatin to the compound of formula (I) or a pharmaceutically acceptable salt thereof per weekly dose is about 1.4:1 to about 32:1.
Paragraph 5. The pharmaceutical combination according to Paragraph 1 or Paragraph 2, wherein the molar ratio of carboplatin to the compound of formula (I) or a pharmaceutically acceptable salt thereof per weekly dose is about 3.3:1 to about 14:1.
Paragraph 6. A method for treating a neoplastic disease in a subject in need thereof, in particular a human, comprising administering to the subject a therapeutically effective amount of a pharmaceutical combination as defined in any one of Paragraphs 1 to 5.
Paragraph 7. The method according to Paragraph 6, wherein carboplatin is administered to the subject before the compound of formula (I) or a pharmaceutically acceptable salt thereof is administered to the subject.
Paragraph 8. The method according to Paragraph 6, wherein carboplatin is administered to the subject at least about 30 minutes before the compound of formula (I) or a pharmaceutically acceptable salt thereof is administered to the subject.
Paragraph 9. The method according to Paragraph 6, wherein carboplatin is administered to the subject at least about one hour before the compound of formula (I) or a pharmaceutically acceptable salt thereof is administered to the subject.
Paragraph 10. The method according to Paragraph 6, wherein carboplatin is administered to the subject at least about four hours before the compound of formula (I) or a pharmaceutically acceptable salt thereof is administered to the subject.
Paragraph 11. The method according to Paragraph 6, wherein when carboplatin and the compound of formula (I) or a pharmaceutically acceptable salt thereof are both administered to the subject within a 48 hour period then carboplatin is administered to the subject about 30 minutes to about 24 hours before the compound of formula (I) or a pharmaceutically acceptable salt thereof is administered to the subject.
Paragraph 12. The method according to any one of Paragraphs 6 to 11, wherein the compound of formula (I) or a pharmaceutically acceptable salt thereof and carboplatin are administered intravenously to the subject.
Paragraph 13. The method according to any one of Paragraphs 6 to 12, wherein the compound of formula (I) or a pharmaceutically acceptable salt thereof and carboplatin are administered to the subject according to a three week treatment cycle (preferably the same three week treatment cycle), wherein the compound of formula (I) or a pharmaceutically acceptable salt thereof is administered in week one of the treatment cycle followed by two rest weeks and carboplatin is administered in week one of the treatment cycle followed by two rest weeks.
Paragraph 14. The method according to Paragraph 13, wherein the compound of formula (I) or a pharmaceutically acceptable salt thereof is administered on day 1 of the treatment cycle and carboplatin is administered on day 1 of the treatment cycle.
Paragraph 15. The method according to any one of Paragraphs 6 to 12, wherein the compound of formula (I) or a pharmaceutically acceptable salt thereof and carboplatin are administered to the subject according to a three week treatment cycle (preferably the same three week treatment cycle), wherein the compound of formula (I) or a pharmaceutically acceptable salt thereof is administered in week one and in week two of the treatment cycle, with week three being a rest week, and carboplatin is administered in week one of the treatment cycle followed by two rest weeks.
Paragraph 16. The method according to Paragraph 15, wherein the compound of formula (I) or a pharmaceutically acceptable salt thereof is administered on days 1 and 8 of the treatment cycle and carboplatin is administered on day 1 of the treatment cycle.
Paragraph 17. The method according to any one of Paragraphs 6 to 16, wherein the molar ratio of carboplatin to the compound of formula (I) or a pharmaceutically acceptable salt thereof per weekly dose is about 0.6:1 to about 250:1.
Paragraph 18. The method according to any one of Paragraphs 6 to 16, wherein the molar ratio of carboplatin to the compound of formula (I) or a pharmaceutically acceptable salt thereof per weekly dose is about 3.3:1 to about 14:1.
Paragraph 19. The method according to any one of Paragraphs 6 to 16, wherein the molar ratio of carboplatin to the compound of formula (I) or a pharmaceutically acceptable salt thereof per weekly dose is about 3.3:1 to about 14:1.
Paragraph 20. The method according to any one of Paragraphs 6 to 16, wherein the compound of formula (I) or a pharmaceutically acceptable salt thereof is administered to a patient at a dose corresponding to the mole equivalent of about 5 mg to about 480 mg of the free base of the compound of formula (I) per week in weeks when administered.
Paragraph 21. The method according to any one of Paragraphs 6 to 16, wherein the compound of formula (I) or a pharmaceutically acceptable salt thereof is administered to a patient at a dose corresponding to the mole equivalent of about 40 mg to about 200 mg of the free base of the compound of formula (I) per week in weeks when administered.
Paragraph 22. The method according to any one of Paragraphs 6 to 16, wherein the compound of formula (I) or a pharmaceutically acceptable salt thereof is administered to a patient at a dose corresponding to the mole equivalent of about 80 mg to about 160 mg of the free base of the compound of formula (I) per week in weeks when administered.
Paragraph 23. The method according to any one of Paragraphs 6 to 16, wherein the compound of formula (I) or a pharmaceutically acceptable salt thereof is administered to a patient at a dose corresponding to the mole equivalent of about 90 mg to about 130 mg of the free base of the compound of formula (I) per week in weeks when administered.
Paragraph 24. The method according to any one of Paragraphs 6 to 16, wherein the compound of formula (I) or a pharmaceutically acceptable salt thereof is administered to a patient at a dose corresponding to the mole equivalent of about 140 mg to about 240 mg of the free base of the compound of formula (I) per week in weeks when administered.
Paragraph 25. The method according to any one of Paragraphs 6 to 16, wherein the compound of formula (I) or a pharmaceutically acceptable salt thereof is administered to a patient at a dose corresponding to the mole equivalent of about 160 mg to about 220 mg of the free base of the compound of formula (I) per week in weeks when administered.
Paragraph 26. The method according to any one of Paragraphs 6 to 16, wherein the compound of formula (I) or a pharmaceutically acceptable salt thereof is administered to a patient at a dose corresponding to the mole equivalent of about 180 mg to about 200 mg of the free base of the compound of formula (I) per week in weeks when administered.
Paragraph 27. The method according to any one of Paragraphs 17 to 26, wherein carboplatin is administered to a patient according to AUC4 per week in weeks when administered.
Paragraph 28. The method according to any one of Paragraphs 17 to 26, wherein carboplatin is administered to a patient according to AUC5 per week in weeks when administered.
Paragraph 29. The method according to any one of Paragraphs 17 to 26, wherein carboplatin is administered to a patient according to AUC6 per week in weeks when administered.
Paragraph 30. The method according to any one of Paragraphs 6 to 29, wherein the neoplastic disease is a solid tumor.
Paragraph 31. The method according to any one of Paragraphs 6 to 30, wherein the neoplastic disease is selected from the group consisting of epithelial neoplasms, squamous cell neoplasms, basal cell neoplasms, transitional cell papillomas and carcinomas, adenomas and adenocarcinomas, adnexal and skin appendage neoplasms, mucoepidermoid neoplasms, cystic neoplasms, mucinous and serous neoplasms, ducal-, lobular and medullary neoplasms, acinar cell neoplasms, complex epithelial neoplasms, specialized gonadal neoplasms, paragangliomas and glomus tumors, naevi and melanomas, soft tissue tumors and sarcomas, fibromatous neoplasms, myxomatous neoplasms, lipomatous neoplasms, myomatous neoplasms, complex mixed and stromal neoplasms, fibroepithelial neoplasms, synovial like neoplasms, mesothelial neoplasms, germ cell neoplasms, trophoblastic neoplasms, mesonephromas, blood vessel tumors, lymphatic vessel tumors, osseous and chondromatous neoplasms, giant cell tumors, miscellaneous bone tumors, odontogenic tumors, gliomas, neuroepitheliomatous neoplasms, meningiomas, nerve sheath tumors, granular cell tumors and alveolar soft part sarcomas, Hodgkin's and non-Hodgkin's lymphomas, other lymphoreticular neoplasms, plasma cell tumors, mast cell tumors, immunoproliferative diseases, leukemias, miscellaneous myeloproliferative disorders, lymphoproliferative disorders and myelodysplastic syndromes.
Paragraph 32. The method according to any one of Paragraphs 6 to 31, wherein the neoplastic disease is one which is treatable by inhibition of PLK1 in addition to a treatment with carboplatin and a TTK inhibitor (e.g. the compound of formula (I)).
Paragraph 33. The method according to any one of Paragraphs 6 to 32, wherein the neoplastic disease is cancer.
Paragraph 34. The method according to Paragraph 33, wherein the cancer in terms of the organs and parts of the body affected is selected from brain, breast (including triple negative breast cancer), cervix, ovaries, colon, rectum (including colon and rectum i.e. colorectal cancer), lung (including small cell lung cancer, non-small cell lung cancer, large cell lung cancer and mesothelioma), endocrine system, bone, adrenal gland, thymus, liver, stomach, intestine (including gastric cancer), pancreas, bone marrow, hematological malignancies (such as lymphoma, leukemia, myeloma or lymphoid malignancies), bile duct, bladder, urinary tract, kidneys, skin, thyroid, head, neck, prostate and testis.
Paragraph 35. The method according to Paragraph 33, wherein the cancer is selected from breast cancer (including triple negative breast cancer), gastric cancer, colorectal cancer, liver cancer (including hepatocellular cancer), endometrial cancer, ovarian cancer, esophageal cancer, lung cancer (including small cell lung cancer, non-small cell lung cancer), Kaposi's sarcoma, cervical cancer, pancreatic cancer, melanoma, prostate cancer, testicular cancer, cervical cancer, bladder cancer, head and neck cancer, brain tumor (e.g. glioma, medulloblastoma), neuroblastoma, retinoblastoma, Wilms' tumor, leukemia, e.g. acute myeloid leukemia (AML) (including Complex Karyotype AML) and malignant mesothelioma.
Paragraph 36. The method according to Paragraph 33, wherein the cancer is breast cancer, e.g. triple negative breast cancer.
Paragraph 37. The method according to Paragraph 33, wherein the cancer is ovarian cancer.
Paragraph 38. A method for treating a neoplastic disease in a subject in need thereof comprising administering to the subject a therapeutically effective amount of the compound of formula (I) or a pharmaceutically acceptable salt thereof, wherein said subject is undergoing or will undergo treatment with carboplatin.
Paragraph 39. The method according to Paragraph 38, wherein the compound of formula (I) or a pharmaceutically acceptable salt thereof is administered to the subject as defined in any one of Paragraphs 7 to 29.
Paragraph 40. The method according to Paragraph 38 or Paragraph 39, wherein the neoplastic disease is as defined in any one of Paragraphs 30 to 37.
Paragraph 41. A method for treating a neoplastic disease in a subject in need thereof comprising administering to the subject a therapeutically effective amount of carboplatin, wherein said subject is undergoing or will undergo treatment with the compound of formula (I) or a pharmaceutically acceptable salt thereof.
Paragraph 42. The method according to Paragraph 41, wherein carboplatin is administered to the subject as defined in any one of Paragraphs 7 to 29.
Paragraph 43. The method according to Paragraph 41 or Paragraph 42, wherein the neoplastic disease is as defined in any one of Paragraphs 30 to 37.
Paragraph 44. A pharmaceutical combination as defined in any one of Paragraphs 1 to 5, for use in the treatment of a neoplastic disease in a subject, in particular a human.
Paragraph 45. The pharmaceutical combination for use according to Paragraph 44, wherein carboplatin is administered to the subject before the compound of formula (I) or a pharmaceutically acceptable salt thereof is administered to the subject.
Paragraph 46. The pharmaceutical combination for use according to Paragraph 44, wherein carboplatin is administered to the subject at least about 30 minutes before the compound of formula (I) or a pharmaceutically acceptable salt thereof is administered to the subject.
Paragraph 47. The pharmaceutical combination for use according to Paragraph 44, wherein carboplatin is administered to the subject at least about one hour before the compound of formula (I) or a pharmaceutically acceptable salt thereof is administered to the subject.
Paragraph 48. The pharmaceutical combination for use according to Paragraph 44, wherein carboplatin is administered to the subject at least about four hours before the compound of formula (I) or a pharmaceutically acceptable salt thereof is administered to the subject.
Paragraph 49. The pharmaceutical combination for use according to Paragraph 44, wherein when carboplatin and the compound of formula (I) or a pharmaceutically acceptable salt thereof are both administered to the subject within a 48 hour period then carboplatin is administered to the subject about 30 minutes to about 24 hours before the compound of formula (I) or a pharmaceutically acceptable salt thereof is administered to the subject.
Paragraph 50. The pharmaceutical combination for use according to any one of Paragraphs 44 to 49, wherein the compound of formula (I) or a pharmaceutically acceptable salt thereof and carboplatin are administered intravenously to the subject.
Paragraph 51. The pharmaceutical combination for use according to any one of Paragraphs 44 to 50, wherein the compound of formula (I) or a pharmaceutically acceptable salt thereof and carboplatin are administered to the subject according to a three week treatment cycle (preferably the same three week treatment cycle), wherein the compound of formula (I) or a pharmaceutically acceptable salt thereof is administered in week one of the treatment cycle followed by two rest weeks and carboplatin is administered in week one of the treatment cycle followed by two rest weeks.
Paragraph 52. The pharmaceutical combination for use according to Paragraph 51, wherein the compound of formula (I) or a pharmaceutically acceptable salt thereof is administered on day 1 of the treatment cycle and carboplatin is administered on day 1 of the treatment cycle.
Paragraph 53. The pharmaceutical combination for use according to any one of Paragraphs 44 to 50, wherein the compound of formula (I) or a pharmaceutically acceptable salt thereof and carboplatin are administered to the subject according to a three week treatment cycle (preferably the same three week treatment cycle), wherein the compound of formula (I) or a pharmaceutically acceptable salt thereof is administered in week one and in week two of the treatment cycle, with week three being a rest week, and carboplatin is administered in week one of the treatment cycle followed by two rest weeks.
Paragraph 54. The pharmaceutical combination for use according to Paragraph 53, wherein the compound of formula (I) or a pharmaceutically acceptable salt thereof is administered on days 1 and 8 of the treatment cycle and carboplatin is administered on day 1 of the treatment cycle.
Paragraph 55. The pharmaceutical combination for use according to any one of Paragraphs 44 to 54, wherein the molar ratio of carboplatin to the compound of formula (I) or a pharmaceutically acceptable salt thereof per weekly dose is about 0.6:1 to about 250:1.
Paragraph 56. The pharmaceutical combination for use according to any one of Paragraphs 44 to 54, wherein the molar ratio of carboplatin to the compound of formula (I) or a pharmaceutically acceptable salt thereof per weekly dose is about 3.3:1 to about 14:1.
Paragraph 57. The pharmaceutical combination for use according to any one of Paragraphs 44 to 54, wherein the molar ratio of carboplatin to the compound of formula (I) or a pharmaceutically acceptable salt thereof per weekly dose is about 3.3:1 to about 14:1.
Paragraph 58. The pharmaceutical combination for use according to any one of Paragraphs 44 to 54, wherein the compound of formula (I) or a pharmaceutically acceptable salt thereof is administered to a patient at a dose corresponding to the mole equivalent of about 5 mg to about 480 mg of the free base of the compound of formula (I) per week in weeks when administered.
Paragraph 59. The pharmaceutical combination for use according to any one of Paragraphs 44 to 54, wherein the compound of formula (I) or a pharmaceutically acceptable salt thereof is administered to a patient at a dose corresponding to the mole equivalent of about 40 mg to about 200 mg of the free base of the compound of formula (I) per week in weeks when administered.
Paragraph 60. The pharmaceutical combination for use according to any one of Paragraphs 44 to 54, wherein the compound of formula (I) or a pharmaceutically acceptable salt thereof is administered to a patient at a dose corresponding to the mole equivalent of about 80 mg to about 160 mg of the free base of the compound of formula (I) per week in weeks when administered.
Paragraph 61. The pharmaceutical combination for use according to any one of Paragraphs 44 to 54, wherein the compound of formula (I) or a pharmaceutically acceptable salt thereof is administered to a patient at a dose corresponding to the mole equivalent of about 90 mg to about 130 mg of the free base of the compound of formula (I) per week in weeks when administered.
Paragraph 62. The pharmaceutical combination for use according to any one of Paragraphs 44 to 54, wherein the compound of formula (I) or a pharmaceutically acceptable salt thereof is administered to a patient at a dose corresponding to the mole equivalent of about 140 mg to about 240 mg of the free base of the compound of formula (I) per week in weeks when administered.
Paragraph 63. The pharmaceutical combination for use according to any one of Paragraphs 44 to 54, wherein the compound of formula (I) or a pharmaceutically acceptable salt thereof is administered to a patient at a dose corresponding to the mole equivalent of about 160 mg to about 220 mg of the free base of the compound of formula (I) per week in weeks when administered.
Paragraph 64. The pharmaceutical combination for use according to any one of Paragraphs 44 to 54, wherein the compound of formula (I) or a pharmaceutically acceptable salt thereof is administered to a patient at a dose corresponding to the mole equivalent of about 180 mg to about 200 mg of the free base of the compound of formula (I) per week in weeks when administered.
Paragraph 65. The pharmaceutical combination for use according to any one of Paragraphs 58 to 64, wherein carboplatin is administered to a patient according to AUC4 per week in weeks when administered.
Paragraph 66. The pharmaceutical combination for use according to any one of Paragraphs 58 to 64, wherein carboplatin is administered to a patient according to AUC5 per week in weeks when administered.
Paragraph 67. The pharmaceutical combination for use according to any one of Paragraphs 58 to 64, wherein carboplatin is administered to a patient according to AUC6 per week in weeks when administered.
Paragraph 68. The pharmaceutical combination for use according to any one of Paragraphs 44 to 67, wherein the neoplastic disease is a solid tumor.
Paragraph 69. The pharmaceutical combination for use according to any one of Paragraphs 44 to 68, wherein the neoplastic disease is selected from the group consisting of epithelial neoplasms, squamous cell neoplasms, basal cell neoplasms, transitional cell papillomas and carcinomas, adenomas and adenocarcinomas, adnexal and skin appendage neoplasms, mucoepidermoid neoplasms, cystic neoplasms, mucinous and serous neoplasms, ducal-, lobular and medullary neoplasms, acinar cell neoplasms, complex epithelial neoplasms, specialized gonadal neoplasms, paragangliomas and glomus tumors, naevi and melanomas, soft tissue tumors and sarcomas, fibromatous neoplasms, myxomatous neoplasms, lipomatous neoplasms, myomatous neoplasms, complex mixed and stromal neoplasms, fibroepithelial neoplasms, synovial like neoplasms, mesothelial neoplasms, germ cell neoplasms, trophoblastic neoplasms, mesonephromas, blood vessel tumors, lymphatic vessel tumors, osseous and chondromatous neoplasms, giant cell tumors, miscellaneous bone tumors, odontogenic tumors, gliomas, neuroepitheliomatous neoplasms, meningiomas, nerve sheath tumors, granular cell tumors and alveolar soft part sarcomas, Hodgkin's and non-Hodgkin's lymphomas, other lymphoreticular neoplasms, plasma cell tumors, mast cell tumors, immunoproliferative diseases, leukemias, miscellaneous myeloproliferative disorders, lymphoproliferative disorders and myelodysplastic syndromes.
Paragraph 70. The pharmaceutical combination for use according to any one of Paragraphs 44 to 69 wherein the neoplastic disease one which is treatable by inhibition of PLK1 in addition to a treatment with carboplatin and a TTK inhibitor (e.g. the compound of formula (I)).
Paragraph 71. The pharmaceutical combination for use according to any one of Paragraphs 44 to 70, wherein the neoplastic disease is cancer.
Paragraph 72. The pharmaceutical combination for use according to Paragraph 71, wherein the cancer in terms of the organs and parts of the body affected is selected from the brain, breast (including triple negative breast cancer), cervix, ovaries, colon, rectum (including colon and rectum i.e. colorectal cancer), lung (including small cell lung cancer, non-small cell lung cancer, large cell lung cancer and mesothelioma), endocrine system, bone, adrenal gland, thymus, liver, stomach, intestine (including gastric cancer), pancreas, bone marrow, hematological malignancies (such as lymphoma, leukemia, myeloma or lymphoid malignancies), bile duct, bladder, urinary tract, kidneys, skin, thyroid, head, neck, prostate and testis.
Paragraph 73. The pharmaceutical combination for use according to Paragraph 71, wherein the cancer is selected from breast cancer (including triple negative breast cancer), gastric cancer, colorectal cancer, liver cancer (including hepatocellular cancer), endometrial cancer, ovarian cancer, esophageal cancer, lung cancer (including small cell lung cancer, non-small cell lung cancer), Kaposi's sarcoma, cervical cancer, pancreatic cancer, melanoma, prostate cancer, testicular cancer, cervical cancer, bladder cancer, head and neck cancer, brain tumor (e.g. glioma, medulloblastoma), neuroblastoma, retinoblastoma, Wilms' tumor, leukemia, e.g. acute myeloid leukemia (AML) (including Complex Karyotype AML) and malignant mesothelioma.
Paragraph 74. The pharmaceutical combination for use according to Paragraph 71, wherein the cancer is breast cancer, e.g. triple negative breast cancer.
Paragraph 75. The pharmaceutical combination for use according to Paragraph 71, wherein the cancer is ovarian cancer.
Paragraph 76. A compound of formula (I) or a pharmaceutically acceptable salt thereof for use in combination with carboplatin for the treatment of a neoplastic disease in a subject, in particular a human.
Paragraph 77. The compound of formula (I) or a pharmaceutically acceptable salt thereof for use according to Paragraph 76, wherein the compound of formula (I) or a pharmaceutically acceptable salt thereof is administered to the subject as defined in any one of Paragraphs 45 to 67.
Paragraph 78. The compound of formula (I) or a pharmaceutically acceptable salt thereof for use according to Paragraph 76 or Paragraph 77, wherein the neoplastic disease is as defined in any one of Paragraphs 68 to 75.
Paragraph 79. Carboplatin for use in combination with a compound of formula (I) or a pharmaceutically acceptable salt thereof for the treatment of a neoplastic disease in a subject, in particular a human.
Paragraph 80. Carboplatin for use according to Paragraph 79, wherein carboplatin is administered to the subject as defined in any one of Paragraphs 45 to 67.
Paragraph 81. Carboplatin for use according to Paragraph 80 or Paragraph 81, wherein the neoplastic disease is as defined in any one of Paragraphs 68 to 75.
Paragraph 82. Use of a pharmaceutical combination as defined in any one of Paragraphs 1 to 5 in the preparation of single-agent medicaments or as a combined medicament for the treatment of a neoplastic disease in a subject, in particular a human.
Paragraph 83. The use according to Paragraph 82, wherein the pharmaceutical composition is administered to the subject as defined in any one of Paragraphs 45 to 67.
Paragraph 84. The use according to Paragraph 82 or Paragraph 83, wherein the neoplastic disease is as defined in any one of Paragraphs 68 to 75.
Paragraph 85. Use of a compound of formula (I) or a pharmaceutically acceptable salt thereof in the preparation of a single-agent medicament for use in combination with carboplatin or in the preparation of a combined medicament with carboplatin, for the treatment of a neoplastic disease in a subject, in particular a human.
Paragraph 86. The use according to Paragraph 85, wherein the compound of formula (I) or a pharmaceutically acceptable salt thereof is administered to the subject as defined in any one of Paragraphs 45 to 67.
Paragraph 87. The use according to Paragraph 85 or Paragraph 86, wherein the neoplastic disease is as defined in any one of Paragraphs 68 to 75.
Paragraph 88. Use of carboplatin in the preparation of a single-agent medicament for use in combination with a compound of formula (I) or a pharmaceutically acceptable salt thereof or in the preparation of a combined medicament with the compound of formula (I) or a pharmaceutically acceptable salt thereof, for the treatment of a neoplastic disease in a subject, in particular a human.
Paragraph 89. The use according to Paragraph 88, wherein carboplatin is administered to the subject as defined in any one of Paragraphs 45 to 67.
Paragraph 90. The use according to Paragraph 88 or Paragraph 89, wherein the neoplastic disease is as defined in any one of Paragraphs 68 to 75.
Paragraph 91. A kit comprising a pharmaceutical combination as defined in any one of Paragraphs 1 to 5, wherein component (a) and component (b) are provided as single-agent medicaments.
Paragraph 92. The kit according to Paragraph 91, wherein the kit is for use in treating a neoplastic disease, in particular as defined in any one of Paragraphs 68 to 75.
Paragraph 93. The kit according to Paragraph 91 or Paragraph 92, further comprising instructions for simultaneous, separate or sequential administration thereof for use in the treatment of a neoplastic disease, in particular a cancer, in a subject, in particular a human.

A number of publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Particular embodiments of the invention are described in the following Examples, which serve to illustrate the invention in more detail and should not be construed as limiting the invention in any way.

### Examples

### Methodology

### Kinase Assay

A radiometric protein kinase assay (33PanQinase® Activity Assay) was used for measuring the kinase activity of TTK and PLKL. TTK and PLK1 protein kinases were expressed as recombinant full-length GST-fusion proteins. The reaction cocktail contained 25 L of assay buffer (standard buffer/[γ-³³P]-ATP) and 10 L of ATP solution (in water), 5 L of test compound and 10 L of enzyme/substrate mixture. The assay for the protein kinases contained 70 mM HEPES-NaOH pH 7.5, 3 mM MgCl₂, 3 mM MnCl₂, 3 M Na-orthovanadate, 1.2 mM DTT, 50 g/ml PEG20000, ATP (0.3 M for TTK and 1 M for PLK1), [γ-³³P]-ATP (approx. 8×10⁵ cpm per well), protein kinase (15.8 nM for TTK and 5 nM for PLK1), and substrate (1 g/50 L for TTK and 2 g/50 L for PLK1). All assays were performed with a BeckmanCoulter/SAGIAN™ Core System. The fitting model for the IC50 determinations was “Sigmoidal response (variable slope)” with parameters “top” fixed at 100% and “bottom” at 0%. The fitting method used was a least squares fit.

### Target Residence Time Assays

The affinity, i.e. the equilibrium dissociation constant (KD) (referred to as residence times), of the compound of formula (I) against TTK and PLK1 were determined using a Biacore T200™ surface plasmon resonance instrument using recombinant expressed TTK kinase domain (amino acids 519-808) or biotinylated PLK1. The immobilization of TTK was performed as described in Maia et al. Annals of Oncology, 2015; 26:2180-2192. The immobilization of biotinylated PLK1 was performed as described in Willemsen-Seegers et al. Journal of Molecular Biology, 2017; 429:574-586. The subsequent single cycle kinetic assays were performed at 22° C. using a compound concentration gradient of 1, 3.6, 10, 31.6 and 100 nM for TTK and 10, 31.6, 100, 316 and 1000 nM for PLK1, a contact time of 100 s and a flow rate of 30 L/min. The dissociation period was 1200-1800 s and a correction for an unstable surface using a blank run with buffer was performed. Binding kinetics were calculated based on the binding curves, demonstrating good signal-to-noise ratios for all compounds tested (data not shown).

### PDX Studies

In a first PDX study, female BALB/c nude mice (CrownBio) of at least 20 g body weight were subcutaneously (s.c.) inoculated in the right flank with tumor fragments (3*3*3 mm) from stock mice bearing the patient-derived xenograft (PDX) triple negative breast cancer (TNBC) tumor, BR1282. Randomization into 5 different treatment groups (8 mice per group) was made when the mean tumor size was 150-200 mm³. Mice were dosed intravenously (i.v.) twice weekly (2qw) with the compound of formula (I) (7 mg/kg, i.v., 5 mL/kg) or once weekly (qw) carboplatin (60 mg/kg, i.v., 10 mL/kg) or the combination of these doses, in which the compound of formula (I) was administered after carboplatin using either a 4 h or a 24 h gap, when both compounds were administered on the same day/consecutive days. The fifth group received both vehicles using the time interval of 4 h and all groups were dosed twice via the tail vein. Dosing was delayed by 1 day in any mice showing transient body weight loss of >10% at any time point.

In a second PDX study, the same model was used as for the first study (TNBC BR1282 PDX model), but this time compound of formula (I) (8 mg/kg, i.v., qw, 5 mL/kg) was administered first and carboplatin (60 mg/kg, i.v., qw, 6 mL/kg) second using a 4 h gap between both administrations and compound of formula (I) was provided qw. Consequently, the mice received both compounds on the same day (4 h gap).

Carboplatin was purchased from Qilu Pharmaceutical Co. Ltd. as a solution (10 mg/mL in 5% (w/v) glucose in pure water). The compound of formula (I) was supplied as a powder by Basilea Pharmaceutica International Ltd. and was prepared freshly as a solution in Ethanol:PEG400:Citric acid 20 mM, at a ratio of 1:1:8 and at a final pH of 3.7, for immediate administration.

Body weights and tumor volumes were determined at least twice per week, the latter by measuring two dimensions with calipers and applying the formula “V=(L×W²)/2”, where V is the tumor volume, and L and W are the tumor length and width re-spectively. Individual mice were culled when tumors reached 1500 mm3 or more, or when the body weight loss (BWL) was found to have exceeded 20%. Mice were also culled if the BWL was determined to be >15% for 3 consecutive days. Any mice with >10% BWL, automatically were dosed on one of the following days, as soon as the BWL returned to <10%. All animal protocols were reviewed and approved by the relevant local committees in China, where the studies were performed.

The statistical significance of efficacy were determined in two different ways using a) the endpoint on day 10 or day 14 depending on the study when it was necessary to cull the vehicle group because of tumor volume exceeding 1500 mm³, and b) at the endpoint of day 31 in the first study when exponential curve fits were applied to estimate the rates of tumor growth in the different groups. The degree of an effect was quantified by the delta (A) treated/control ratio (ΔT/C) and the statistical significance of the effect was determined by a one-way analysis of variance (1W-ANOVA) with Holms-Sidak applied post-hoc for multiple determinations, where a p-value of <0.05 was considered statistically significant. In addition, an assessment of the combination effect was made based upon the Clarkes Combination Index (CCI), where the CCI=ΔT/CAB−(ΔT/CA*ΔT/CB) in which A and B are two different compounds and AB the combination thereof, where CCI<−0.1 indicates synergy (or a positive interaction), +0.1 (antagonism, or a negative interaction) and between −0.1 and +0.1 indicates additivity (no interaction); see O'Reilly et al. Anti Cancer Drugs 2011; 22:58-78.

### Results

TTK and PLK1 are kinases with an essential role in control of the spindle assembly checkpoint (SAC), which is a cell cycle surveillance mechanism ensuring optimal cell division via proper chromosome alignment. TTK and PLK1 co-operate to recruit SAC components to the SAC protein complex at the kinetochore of the chromosome, thus inhibition of both enzymes should maximize progression of mitosis via more rapid breakage of the SAC (Von Schubert et al., Cell Reports 2015, 12; 66-78). This has proven to be the case in tumor cell systems when comparing the compound of formula (I) with TKK inhibitors not having any meaningful PLK1 inhibitory activity (data not shown).

In general, the compound of formula (I) shows strong specificity for TTK, with other kinase IC50s being at least 10-fold higher than those for TTK. The TTK kinase assay described above confirmed that the compound of formula (I) is highly potent against TTK, giving an IC50 of 7 nM (0.4 nM when measured as described in WO 2015/155042).

The PLK1 kinase assay described above also showed that the compound of formula (I) targets PLK1. The compound of formula (I) was found to inhibit PLK1 with an IC50 of 72 nM. When measured as described in WO 2015/155042 the compound of formula (I) inhibited PLK1 with an IC50 of 46 nM. Other TTK inhibitors reported in the literature have similar or slightly better overall specificity for TTK but conversely have little or no activity against PLK1 relative to their activity against TTK (data not shown). Importantly, the compound of formula (I) has a very long target-residency of >12 h on TTK, while that for PLK1 is just a few minutes. This prolonged inhibition of TTK combined with a transient effect on PLK1 leads to a rapid disruption of the SAC leaving the cells without adequate time for correct chromosome segregation.

Prolonged TTK target occupancy was also measured in tumors derived from MDA-MB-231 xenograft-bearing mice treated with intermittent i.v. dosing regimen of compound of formula (I). To determine the tumor TTK target occupancy time of the compound of formula (I), mice harboring the TNBC xenograft model MDA-MB-231 were treated i.v. twice-weekly with MTD and sub-MTD doses of the compound of formula (I). Analysis of vehicle- and compound of formula (I)-treated tumors for TTK target occupancy indicated that the compound of formula (I) occupied tumor-derived TTK in a concentration-dependent manner; TTK was completely occupied by the compound of formula (I) for at least 72 h after administration of the last MTD dose. A repeat experiment using i.v., weekly MTD dosing indicated that tumor-derived TTK was fully drug-occupied for up to 6 days after the last administration.

In cellular systems, the compound of formula (I) has high potency against sensitive cells. In a 5-day anti-proliferative screen of 18 different triple negative breast cancer cell lines, the compound of formula (I) had a median GI50 of 35 nM. In mice, the compound of formula (I) has shown significant activity against patient-derived tumor xenografts, including TNBC and hepatocellular cancer (HCC) models, with effects ranging from minimal to very strong including substantial regression (data not shown).

Carboplatin is standard of care treatment in a number of tumor indications. To determine the combination effect of the compound of formula (I) and carboplatin, the maximum tolerated dose (MTD) of the compound of formula (I) in combination with an optimal dose of carboplatin was first defined in non-tumor-bearing BALB/c nude mice. Carboplatin (60 mg/kg, qw, i.v.) was administered first, followed by the compound of formula (I) 24 hours (sequential approach) or 4 hours (near-concomitant approach) later. The highest tolerated doses of the compound of formula (I), administered 2qw, i.v. in combination with optimal dose of carboplatin were 6-7 mg/kg using the schedule described above. In contrast, when compound of formula (I) was applied first and carboplatin (60 mg/kg, i.v., qw) 4 or 24 h later, the non-tumor bearing mice only tolerated a weekly administration of compound of formula (I) at 8 mg/kg using a 4 h gap between compound of formula (I) and carboplatin. Thus, the mice were treated always on the same day with compound of formula (I) and carboplatin. A 24 h gap between both administrations or a 2qw schedule of compound of formula (I) was not tolerated using 6-8 mg/kg of compound of formula (I), when compound of formula (I) was applied first. Importantly, these compound of formula (I) doses are known to be efficacious as monotherapy in tumor-bearing mice.

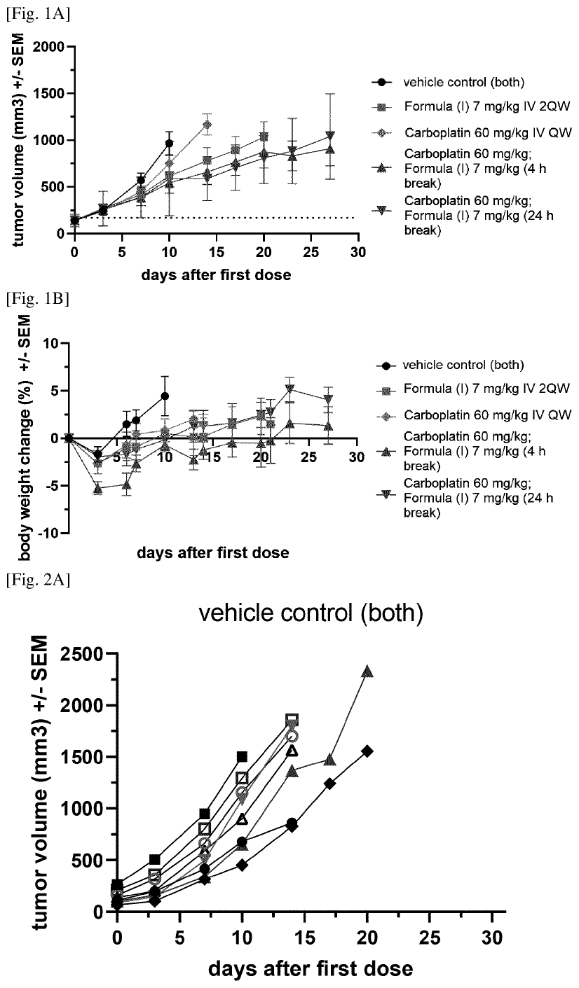

In a first combination experiment, mice harboring BR1282 PDX TNBC tumors were treated with carboplatin at 60 mg/kg qw i.v. and the compound of formula (I) at 7 mg/kg 2qw i.v. (4 or 24 hours later, when administered at the same day or consecutive days). This model was chosen, because it was known to have an intermediate response to the compound of formula (I) and minimal response to carboplatin. As shown before, single agent treatment with either compound was well tolerated with minor or no body weight loss (FIG. 1B). In addition, both combination regimens were well tolerated, although some individual mice had transient body weight loss. Individual mice with body weight loss above 10% were treated one day later than scheduled to allow recovery. The 24 hours gap group tolerated the combination slightly better than the 4 hours gap group, visible by a larger decrease in body weight within one week after treatment start in the latter.

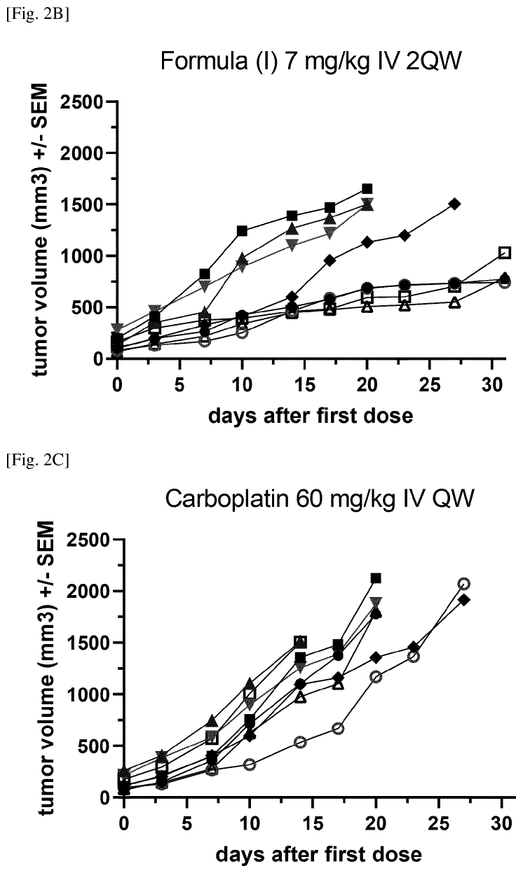

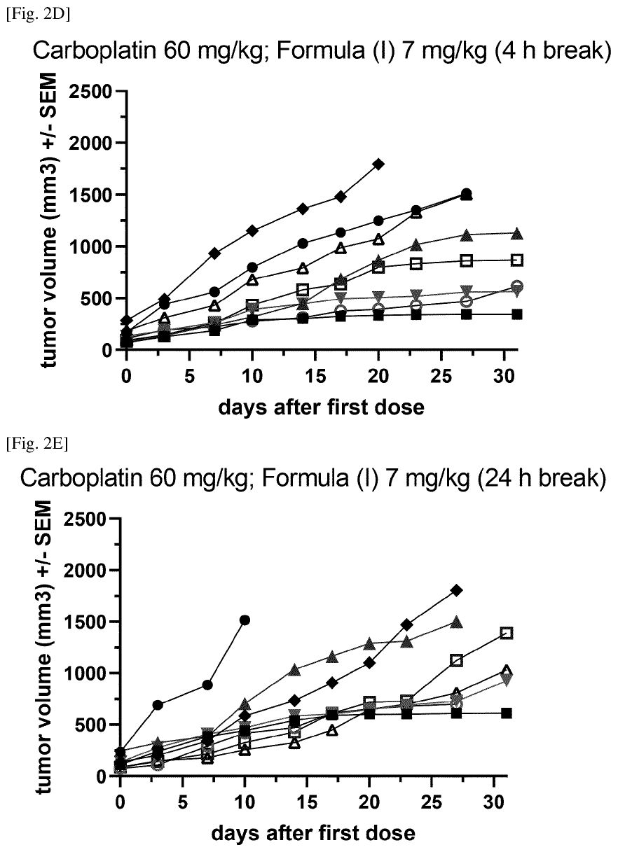

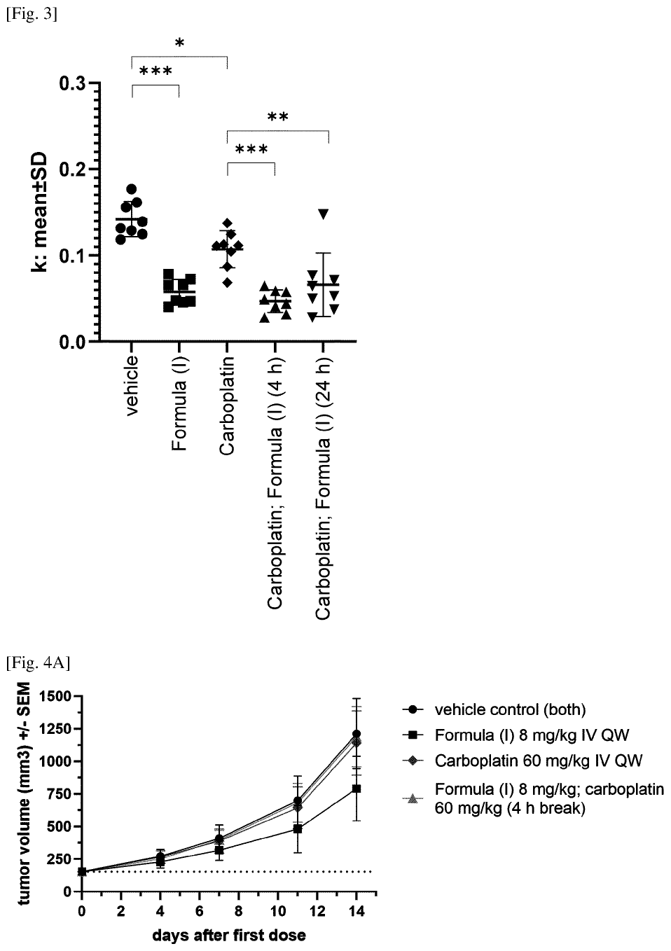

Both combination regimens led to reduced tumor growth, similar to that observed with the compound of formula (I) single agent, whereas very little effect on tumor growth was observed with carboplatin alone (FIGS. 1A and 2). The Clarkes Combination Index (CCI) of approx. 0.1 on treatment day 10 within this study demonstrated a trend for additivity in both combination groups. At that time point, none of the comparisons are significantly different from each other by using the ΔT/C (Table 1). In addition, the treatment is well tolerated as the body weight change on day 10 in the compound-treated groups is not statistically different between groups (p>0.05; Table 1). The treatment of the combination groups could not be extended beyond 31 days due to damaged tail veins as a consequence of the repeated i.v. administrations. Exponential curve fits from day 0 until culling day of each group (latest day 31) provided more statistical power and showed a much stronger effect, so that both combination groups were now highly significantly different to carboplatin monotherapy but not to monotherapy of compound of formula (I) (FIG. 3 and Tables 1 and 2), and the CCI indicated additivity for the 4 h combination group (CCI=0.02) and antagonism for the 24 h combination group (CCI=0.16).

Based on good tolerability and trends for additive effects on tumor growth these data support further evaluation of the compound of formula (I) in combination with carboplatin.

TABLE 1
Summary of efficacy and tolerability in the first combination experiment

| | Efficacy: ΔT/C | | Tolerability (Day 10) | |
|---|---|---|---|---|
| Treatment | Day 10 | Day 0 up to Day 31 | ΔBW (%) | ΔI/C |
| Vehicles | 1.0 | 1.0 | 4.4 ± 2.1 | 1.00 |
| Compound of formula (I) | 0.58 | 0.41*** | 0.5 ± 1.6 | 0.96 |
| Carboplatin | 0.74 | 0.75* | 0.8 ± 1.2 | 0.97 |
| Combination 4 h | 0.49 | 0.33*** | −0.7 ± 0.7 | 0.95 |
| Combination 24 h | 0.54 | 0.46*** | −0.2 ± 1.1 | 0.96 |

Results for efficacy show the calculated ΔT/Cs based on the mean change in tumor volume for each group on day 10 and the mean rate of growth (k) measured from day 0 to day 31. Growth rate (k) is calculated based on the time period for each treatment, i.e. D0-D20 (Vehicle), D0-D27 for carboplatin monotherapy and D0-D31 for the two combination groups as well as the compound of formula (I) monotherapy. All treatments were highly significantly different to the vehicle group, where
***p < 0.001 (one way ANOVA with Holm-Sidak post-hoc), except the carboplatin monotherapy showed a significant
*p value of 0.0167. Results for tolerability show the mean ± SEM for the percentage change in body weight (BW) on day 10 and the ΔT/C based on the mean fractional change compared to the vehicle (BW_{day10}/BW_{day0}).

Table 2 represents the overview of the statistical analysis of mean rate of growth (k) of FIG. 3 for each individual comparison based on one-way-analysis of variance (1WA) with Holm-Sidak post-hoc for group comparisons.

TABLE 2

| | Mean | | Adjusted p |
|---|---|---|---|
| Group comparison | difference | Summary | value |
| Vehicle vs. Formula (I) | 0.084 | *** | <0.001 |
| Vehicle vs. Carboplatin | 0.0350 | * | 0.0167 |
| Vehicle vs. Carboplatin; Formula (I) (4 h) | 0.095 | *** | <0.001 |
| Vehicle vs. Carboplatin; Formula (I) (24 h) | 0.076 | *** | <0.001 |
| Formula (I) vs. Carboplatin | −0.049 | *** | <0.001 |
| Formula (I) vs. Carboplatin; Formula (I) (4 h) | 0.011 | ns | 0.574 |
| Formula (I) vs. Carboplatin; Formula (I) (24 h) | −0.008 | ns | 0.574 |
| Carboplatin vs. Carboplatin; Formula (I) (4 h) | 0.060 | *** | <0.001 |
| Carboplatin vs. Carboplatin; Formula (I) (24 h) | 0.041 | ** | 0.005 |
| Carboplatin; Formula (I) (4 h) vs. Carboplatin; Formula (I) | −0.019 | ns | 0.280 |
| (24 h) | | | |

“*” means p < 0.05,
“**” means p < 0.01,
“***” means p < 0.001 (one way ANOVA with Holm-Sidak post-hoc)

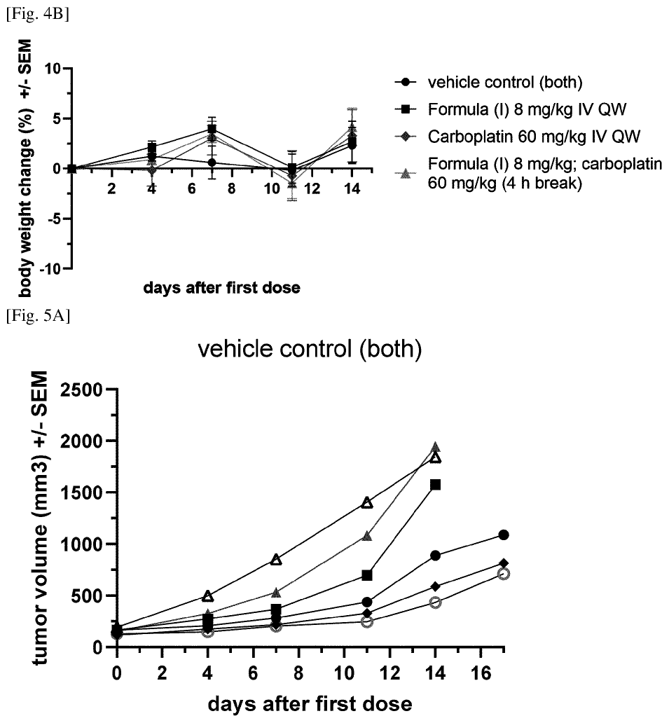

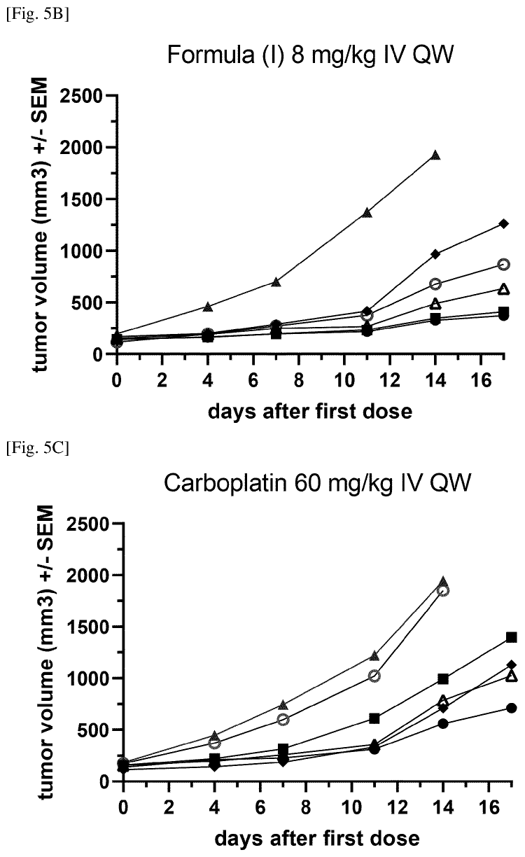

In the second combination study of the same model, compound of formula (I) (8 mg/kg, qw, i.v., 5 mL/kg) was administered first and 4 h later carboplatin (60 mg/kg, qw, i.v., 6 mL/kg). All groups, vehicle control group as well as the compound-treated groups, reached the ethical endpoint on day 14. At that time point it was apparent that only the compound of formula (I) group had some level of efficacy (FIGS. 4A, 5 and 6, Table 3), but this was not statistically significant (all comparisons p>0.05). After exponential growth equation, the differences between the groups were still not statistically significant (Table 3). The CCIs indicated a negative interaction, also called antagonism, with a value of 0.42. Overall, the treatments were well tolerated with no significant differences in body weight change for the treatment groups compared to the vehicle group at day 14 (FIG. 4B and Table 3). In conclusion, when the mice received the compound of formula (I) first and 4 h later carboplatin an antagonism occurs in contrast to the additive effect observed in the first study, when carboplatin was applied 4 h before compound of formula (I). Thus, it is highly important to treat first carboplatin and afterwards compound of formula (I) to receive at least an additive effect.

TABLE 3
Summary of efficacy and tolerability in the second combination experiment

| | Efficacy: ΔT/C | | Tolerability (Day 14) | |
|---|---|---|---|---|
| Treatment | Day 14 | Day 0 up to Day 17 | ΔBW (%) | ΔT/C |
| Vehicles | 1.0 | 1.0 | 2.3 ± 1.8 | 1.0 |
| Compound of formula (I) | 0.60 | 0.70 | 2.7 ± 2.1 | 1.00 |
| Carboplatin | 0.93 | 0.96 | 3.3 ± 2.6 | 1.00 |
| Combination 4 h | 0.98 | 0.94 | 4.2 ± 1.9 | 1.00 |

Results for efficacy show the calculated ΔT/Cs based on the mean change in tumor volume for each group on day 14 and the mean rate of growth (k) measured from day 0 to day 17. Growth rate (k) is calculated based on the time period for each treatment, i.e. D0-D17 for all groups. All comparisons were not significantly different to each other, where p > 0.05 (one way ANOVA with Holm-Sidak post-hoc). Results for tolerability show the mean ± SEM for the percentage change in body weight (BW) on day 14 and the ΔT/C based on the mean fractional change compared to the vehicle (BW_{day14}/BW_{day0}).

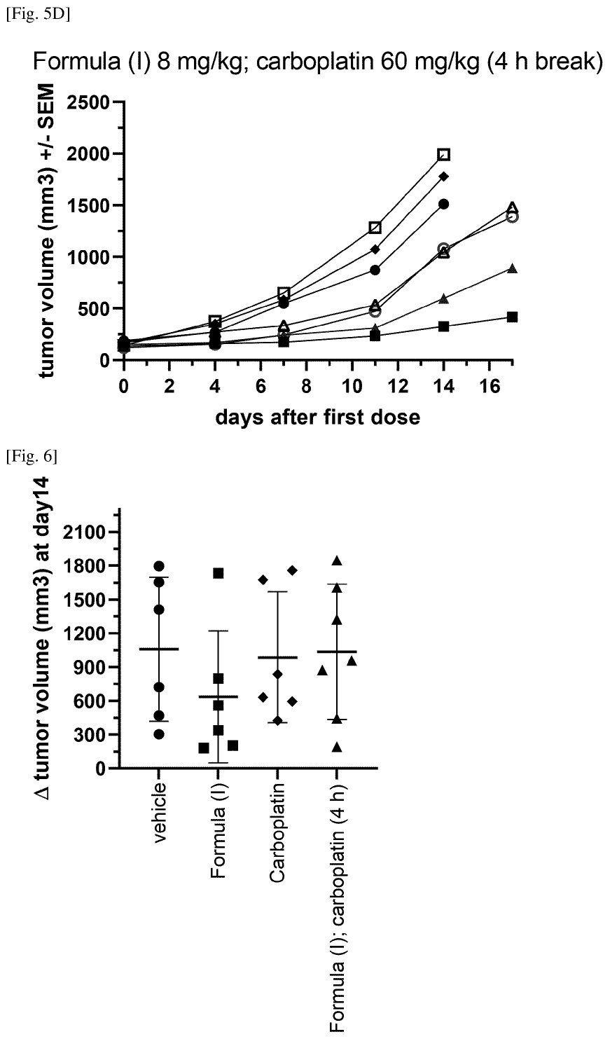

Table 4 represents the overview of the statistical analysis of A tumor volume on day 14 of FIG. 6 and mean rate of growth (k) for each individual comparison based on one-way-analysis of variance (1WA) with Holm-Sidak post-hoc for group comparisons.

TABLE 4

| | Δ tumor volume on day 14 | | k means | |
|---|---|---|---|---|
| | Mean | Adjusted p | Mean | Adjusted |
| Group comparison | difference | value | difference | p value |
| Vehicle vs. Formula (I) | 422.3 | 0.805 | 0.045 | 0.135 |
| Vehicle vs. Carboplatin | 71.5 | 0.996 | 0.007 | 0.947 |
| Vehicle vs. Formula (I); carboplatin (4 h) | 23.2 | 0.996 | 0.009 | 0.947 |
| Formula (I) vs. Carboplatin | −350.8 | 0.805 | −0.039 | 0.223 |
| Formula (I) vs. Formula (I); carboplatin (4 h) | −399.1 | 0.805 | −0.036 | 0.223 |
| Carboplatin vs. Formula (I); carboplatin (4 h) | −48.3 | 0.996 | 0.002 | 0.947 |

The anti-tumor activity of the compound of formula (I) in combination with carboplatin was evaluated in an ovarian cancer model, using human SKOV-3 cells grown as a solid tumor xenograft subcutaneously (s.c.) in nude mice.

The maximum tolerated dose (MTD) of the compound of formula (I) in combination with an optimal dose of carboplatin was defined in non-tumor bearing BALB/c nude mice in the experiments described above. These studies identified the most effective and tolerated schedule as being the compound of formula (I) administered (7 mg/kg, i.v., 2qw) 4 hr after carboplatin (60 mg/kg, i.v, qw). Thus, in this study, using a human ovarian cell line derived xenograft (CDX) model, that schedule was applied and compared directly with the same order of administration with a 1 hr or 24 hr gap between compounds. In addition, the twice-weekly schedule was compared with a weekly schedule of 7 mg/kg BAL0891 (a weekly dose well below the MTD for compound of formula (I) monotherapy) using a 4 hr gap. Treatments continued until day 24 and tumor volumes and body weight were monitored until day 51.

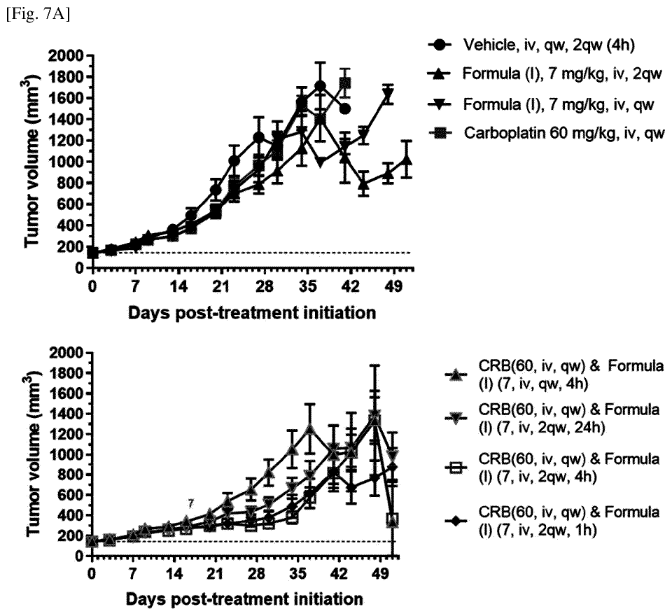

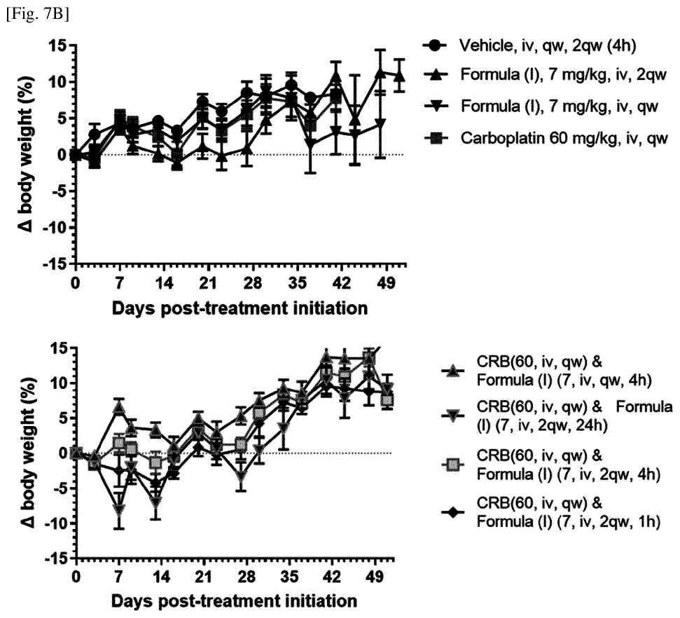

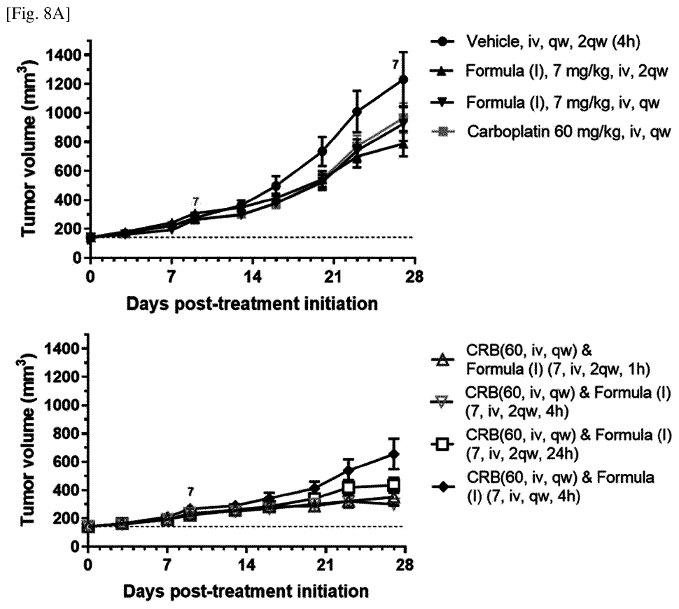

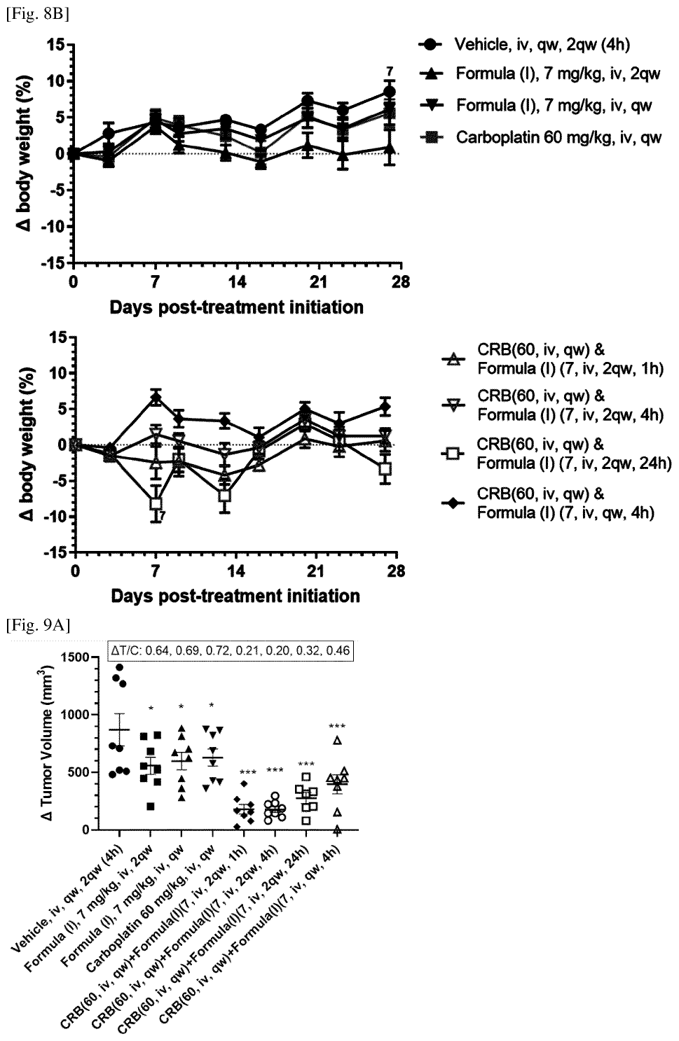

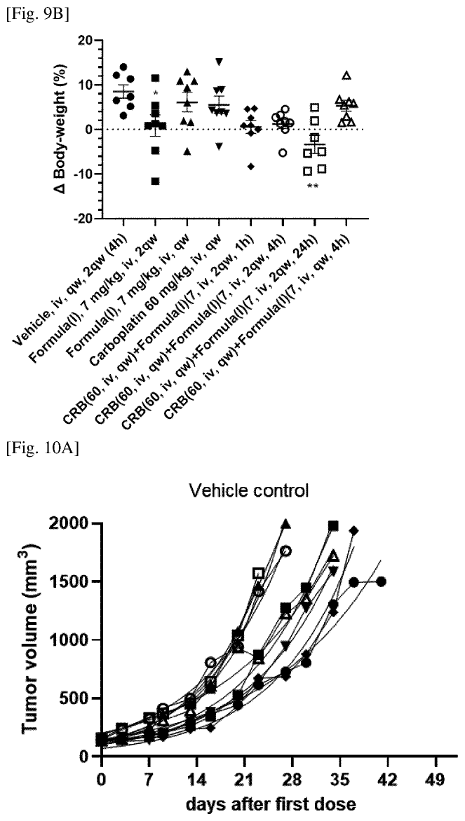

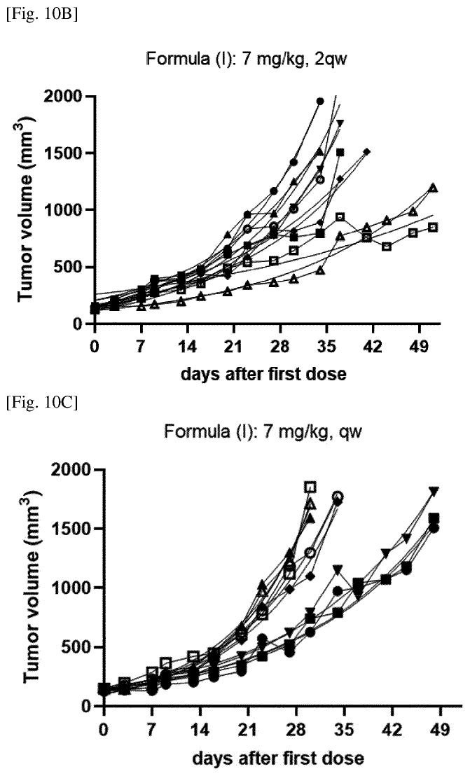

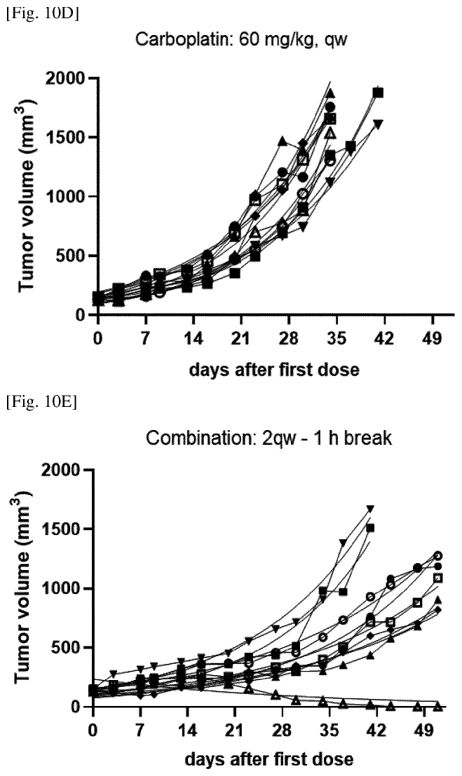

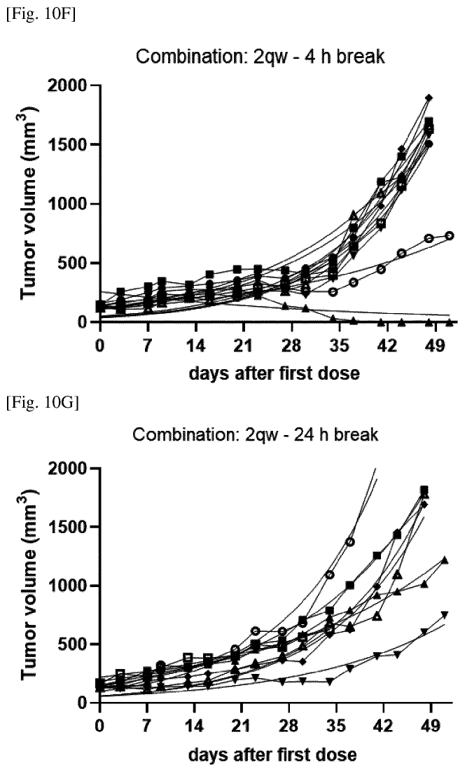

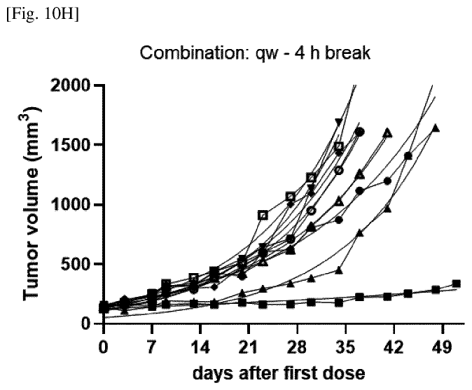

The complete experiment is shown in FIG. 7 which shows the mean±SEM (n=8) for efficacy (TVol) and tolerability (BW) and, from day 23, mice began to be culled when individual tumors reached at least 1500 mm³. For this reason, the chosen endpoint for comparison of efficacy T/Cs was day 23 (FIG. 8, 9, Table 5). Individual plots for each tumor are also shown in FIG. 10, along with exponential fits for tumor growth.

TABLE 5

| Treatments and schedules | ΔT/C | CCI |
|---|---|---|
| Formula (I) (7 mg/kg, iv, 2qw) | 0.64 | Not applicable |
| Formula (I) (7 mg/kg, iv, qw) | 0.69 | Not applicable |
| Carboplatin (60 mg/kg, iv, qw) | 0.72 | Not applicable |
| Carboplatin (60 mg/kg, iv, qw), Formula (I) (7 mg/kg, iv, 2qw); 1 hr | 0.21 | −0.26 |
| Carboplatin (60 mg/kg, iv, qw), Formula (I) (7 mg/kg, iv, 2qw); 4 hr | 0.20 | −0.26 |
| Carboplatin (60 mg/kg, iv, qw), Formula (I) (7 mg/kg, iv, 2qw); 24 hr | 0.32 | −0.15 |
| Carboplatin (60 mg/kg, iv, qw), Formula (I) (7 mg/kg, iv, qw); 1 hr | 0.46 | −0.04 |

CCI: ΔT/C of the combination minus the multiplied sum of the two monotherapy ΔT/Cs; values <−0.1 indicate synergy, while values between −0.1 and +0.1 indicate additivity.

The ΔT/Cs showed only a small anticancer effect for the three different monotherapies, although each was significantly different from the vehicle group on day 23 (p<0.05). However, all the combinations showed highly significant differences to the vehicle (p<0.001) even though the combination of carboplatin with weekly ad-ministration of the compound of formula (I) was clearly the least effective (ΔT/C=0.46). Nevertheless, the multiple comparisons (one-way ANOVA) did not show any significant differences between the combinations at this time-point. Based on day 23 ΔT/Cs, the most effective combinations were carboplatin combined with compound of formula (I) (2qw) using a 1 hr or 4 hr gap between treatments (carboplatin always first) and, indeed, in each of these two groups there was one tumor which completely regressed. This was confirmed on day 51 by immunohistochemistry (IHC) where no tumor cells were visible. An assessment of synergy by the Clarke-Combination-Index (CCI) indicated synergy for the three combinations using BAL0891 administered twice a week (1, 4 or 24 hr interval) while the weekly BAL0891 schedule only gave additivity (Table 5).

All treatments were relatively well tolerated, except for the combination group with a 24 hr gap between carboplatin and compound of formula (I) treatment, in which during the first two weeks there were some marked but transient body-weight losses. Overall, these data suggested significant synergy between the two compounds when shorter intervals (1-4 hr) were utilized since they provided the most efficacy and the best tolerability.

In conclusion, in nude mice bearing the s.c. human ovarian tumor, SKOV-3, the combination of carboplatin (60 mg/kg, iv, qw) administration followed by the compound of formula (I) (7 mg/kg, iv, 2qw) was well tolerated and showed synergistic efficacy. The most effective combinations were carboplatin combined with the compound of formula (I) (2qw) using a 1 hr or 4 hr gap between treatments. Indeed, in each of these two groups there was one tumor which completely regressed. Assessment of synergy by the Clarke-Combination-Index (CCI) indicated synergy for the three combinations using the compound of formula (I) administered twice a week (1, 4 or 24 hr interval). However, the low-dose, weekly compound of formula (I) schedule gave only additivity.

1. A pharmaceutical combination comprising
(a) a compound of formula (I) 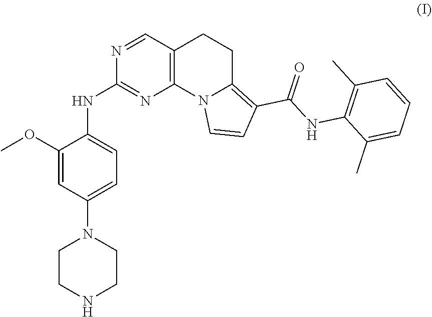 [SMILES: COc1cc(N2CCNCC2)ccc1Nc1ncc2c(n1)-n1ccc(C(=O)Nc3c(C)cccc3C)c1CC2]
or a pharmaceutically acceptable salt thereof;
and (b) carboplatin.

2. The pharmaceutical combination according to claim 1, wherein the molar ratio of carboplatin to the compound of formula (I) or a pharmaceutically acceptable salt thereof per weekly dose is about 2.1:1 to about 32:1.

3. A method for treating a neoplastic disease in a subject, comprising:
administering to the subject the pharmaceutical combination of claim 1.

4. The method according to claim 3, wherein carboplatin is administered to the subject before the compound of formula (I) or a pharmaceutically acceptable salt thereof is administered to the subject.

5. The method according to claim 3, wherein when carboplatin and the compound of formula (I) or a pharmaceutically acceptable salt thereof are both administered to the subject within a 48 hour period then carboplatin is administered to the subject up to 24 hours before the compound of formula (I) or a pharmaceutically acceptable salt thereof is administered to the subject.

6. The method according to claim 3, wherein when carboplatin and the compound of formula (I) or a pharmaceutically acceptable salt thereof are both administered to the subject within a 24 hour period then carboplatin is administered to the subject up to 6 hours before the compound of formula (I) or a pharmaceutically acceptable salt thereof is administered to the subject.

7. The method according to claim 3, wherein when carboplatin and the compound of formula (I) or a pharmaceutically acceptable salt thereof are both administered to the subject within a 48 hour period then carboplatin is administered to the subject about 30 minutes to about 24 hours before the compound of formula (I) or a pharmaceutically acceptable salt thereof is administered to the subject.

8. The method according to claim 3, wherein the compound of formula (I) or a pharmaceutically acceptable salt thereof and carboplatin is administered to the subject according to a three week treatment cycle;
wherein the compound of formula (I) or a pharmaceutically acceptable salt thereof is administered in week one of the treatment cycle followed by two rest weeks and carboplatin is administered in week one of the treatment cycle followed by two rest weeks; or
wherein the compound of formula (I) or a pharmaceutically acceptable salt thereof is administered in week one and in week two of the treatment cycle, with week three being a rest week, and carboplatin is administered in week one of the treatment cycle followed by two rest weeks;
preferably wherein in each case the compound of formula (I) or a pharmaceutically acceptable salt thereof and carboplatin are administered to the subject according to the same three week treatment cycle.

9. The method according to claim 3, wherein the compound of formula (I) or a pharmaceutically acceptable salt thereof is administered to a patient at a dose corresponding to the mole equivalent of about 40 mg to about 200 mg of the free base of the compound of formula (I) per week in weeks when administered.

10. The method according to claim 3, wherein the compound of formula (I) or a pharmaceutically acceptable salt thereof is administered to a patient at a dose corresponding to the mole equivalent of about 80 mg to about 160 mg of the free base of the compound of formula (I) per week in weeks when administered.

11. The method according to claim 3, wherein carboplatin is administered to a patient at a dose according to AUC5 per week in weeks when administered; or wherein carboplatin is administered to a patient at a dose according to AUC6 per week in weeks when administered.

12. The method according to claim 3, wherein the neoplastic disease is selected from the group consisting of epithelial neoplasms, squamous cell neoplasms, basal cell neoplasms, transitional cell papillomas and carcinomas, adenomas and adenocarcinomas, adnexal and skin appendage neoplasms, mucoepidermoid neoplasms, cystic neoplasms, mucinous and serous neoplasms, ducal-, lobular and medullary neoplasms, acinar cell neoplasms, complex epithelial neoplasms, specialized gonadal neoplasms, paragangliomas and glomus tumors, naevi and melanomas, soft tissue tumors and sarcomas, fibromatous neoplasms, myxomatous neoplasms, lipomatous neoplasms, myomatous neoplasms, complex mixed and stromal neoplasms, fibroepithelial neoplasms, synovial like neoplasms, mesothelial neoplasms, germ cell neoplasms, trophoblastic neoplasms, mesonephromas, blood vessel tumors, lymphatic vessel tumors, osseous and chondromatous neoplasms, giant cell tumors, miscellaneous bone tumors, odontogenic tumors, gliomas, neuroepitheliomatous neoplasms, meningiomas, nerve sheath tumors, granular cell tumors and alveolar soft part sarcomas, Hodgkin's and non-Hodgkin's lymphomas, other lymphoreticular neoplasms, plasma cell tumors, mast cell tumors, immunoproliferative diseases, leukemias, myeloproliferative disorders, lymphoproliferative disorders and myelodysplastic syndromes.

13. The method according to claim 3, wherein the neoplastic disease is one which is treatable by inhibition of PLK1 in addition to treatment with carboplatin and a TTK inhibitor.

14. The method according to claim 3, wherein the neoplastic disease is a cancer, in particular a cancer selected from breast cancer, gastric cancer, colorectal cancer, liver cancer, endometrial cancer, ovarian cancer, esophageal cancer, lung cancer, Kaposi's sarcoma, cervical cancer, pancreatic cancer, melanoma, prostate cancer, testicular cancer, cervical cancer, bladder cancer, head and neck cancer, brain tumor, neuroblastoma, retinoblastoma, Wilms' tumor, leukemia, e.g. acute myeloid leukemia (AML) and malignant mesothelioma.

15. The method according to claim 3, wherein the neoplastic disease is breast cancer.

16. The method according to claim 3, wherein the neoplastic disease is triple negative breast cancer.

17. The method according to claim 3, wherein the neoplastic disease is a cancer which is treatable with carboplatin.

18. The method according claim 3, wherein the neoplastic disease is ovarian cancer, lung cancer, cervical cancer, Wilms' Tumor, brain tumors neuroblastoma, testicular cancer, bladder cancer, retinoblastoma, breast cancer, endometrial cancer.

19. The method according to claim 3, wherein the neoplastic disease is ovarian cancer.

20.-21. (canceled)
